(19) United States
(12) Patent Application Publication
(10) Pub. No.: US 2022/0008430 A1
(43) Pub. Date: Jan. 13, 2022

(54) METHODS OF TREATING SUBJECTS HAVING PLATELET DYSFUNCTION WITH IV MELOXICAM

(71) Applicant: BAUDAX BIO, INC., Malvern, PA (US)

(72) Inventors: Randall J. MACK, Malvern, PA (US); Stewart MCCALLUM, Malvern, PA (US)

(21) Appl. No.: 17/292,403

(22) PCT Filed: Nov. 7, 2019
(86) PCT No.: PCT/US19/60278

§ 371 Date: May 7, 2021

Related U.S. Application Data
(60) Provisional application No. 62/757,003, filed on Nov. 7, 2018.

(51) Int. Cl. A61K 31/5415; A61K 45/06; A61P 7/02

(52) U.S. Cl. CPC A61K 31/5415; A61P 7/02; A61K 45/06

(57) ABSTRACT

The disclosure provides methods of treating pain in a first subject in need thereof, comprising administering meloxicam to the first subject, wherein the first subject has a platelet dysfunction. In some embodiments, a closure time of platelets isolated from the first subject before administration of meloxicam is more prolonged than a closure time of platelets isolated from an otherwise similar subject without platelet dysfunction. In some embodiments, the closure time of platelets isolated from the first subject after administration of meloxicam is comparable to a closure time of platelets isolated from the first subject before administration of meloxicam.

### CROSS-REFERENCE TO RELATED APPLICATIONS

This application claims the benefit of priority under 35 U.S.C. § 119(e) of U.S. Provisional Patent Application No. 62/757,003, filed Nov. 7, 2018, the contents of which are incorporated herein by reference in its entirety for all purposes.

### FIELD OF THE DISCLOSURE

The present disclosure relates to methods of administering meloxicam for treatment of pain to subjects with platelet dysfunction.

### BACKGROUND

Meloxicam (4-hydroxy-2-methyl-N-(5-methyl-2-thiazolyl)-2H-1,2-benzothiazine-3-carboxamide-1,1-dioxide) is a long-acting nonsteroidal anti-inflammatory drug (NSAID) that possesses anti-inflammatory, analgesic, and antipyretic activities, which are believed to be related to the inhibition of cyclooxygenase (COX) and subsequent reduction in prostaglandin biosynthesis. Meloxicam has been marketed by Boehringer Ingelheim Pharmaceuticals, Inc. since the 1990's as an oral agent, Mobic®. Mobic is used for treatment of symptoms of osteoarthritis and rheumatoid arthritis.

However, oral meloxicam has a slow onset of action, largely due to poor water solubility, and is not currently approved for the treatment of acute pain. The oral form has a prolonged absorption time, with the time of maximum observed plasma concentration (tₘₐₓ) being approximately 5-6 hours following oral administration, which is consistent with its poor water solubility.

Intravenous (IV) administration of the NSAID ibuprofen was approved in 2009 for pain management; however, infusion time of 30 minutes is required and it must be administered every 6 hours for treatment of pain. See CALDOLOR® Prescribing Information. In addition, patients receiving IV administration of ibuprofen, ketoroloac and other NSAIDs have suffered from relatively high rates of injection site pain or discomfort (e.g., 14%-29% reported), which prohibits faster administration times. See Gan T J, et al., Clinical Therapeutics, 2015, 37, 368-375; Zhou T J, et al. Anesth Analg. 2001; 92:1569-1575. Because current IV NSAIDs require slow injection times and are not administered pre-operatively (i.e., for preventative treatment), subjects experience significant pain before onset of pain relief. Thus, there is a need for a method of administering meloxicam which can provide a faster onset of action and preventive pain treatment of acute pain (mild to moderate pain and moderate to severe pain).

### SUMMARY

The disclosure provides methods of treating pain in a first subject in need thereof, comprising administering meloxicam to the first subject, wherein the first subject has a platelet dysfunction. In some embodiments, the platelet dysfunction is an inherited platelet dysfunction. In some embodiments, the inherited platelet dysfunction is Von Willebrand disease, Glanzmann disease, Wiskott-Aldrich syndrome, Chédiak-Higashi syndrome, or Bernard-Soulier syndrome.

In some embodiments, the platelet dysfunction is an acquired platelet dysfunction. In some embodiments, the acquired platelet dysfunction is caused by an administration of at least one blood thinning drug to the first subject. In some embodiments, the at least one blood thinning drug is at least one anti-platelet drug. In some embodiments, the at least one anti-platelet drug is aspirin, clopidogrel, dipyridamole or ticlopidine. In some embodiments, the at least one anti-platelet drug is a nonsteroidal anti-inflammatory drug. In some embodiments, the at least one blood thinning drug is at least one anti-coagulant. In some embodiments, the at least one anti-coagulant is warfarin, enoxaparin, heparin, dabigatran, apixaban, betrixaban or rivaroxaban. In some embodiments, the first subject has at least one disease or condition that affects platelet function. In some embodiments, the at least one disease or condition that affects platelet function is cirrhosis, multiple myeloma, kidney disease, systemic lupus erythematosus, a disorder of secretion and thromboxane synthesis or uremia. In some embodiments, the first subject was previously subjected to a cardiopulmonary bypass procedure.

In some embodiments, a clotting time of blood isolated from the first subject before administration of meloxicam is more prolonged than a clotting time of blood isolated from an otherwise similar subject without platelet dysfunction. In some embodiments, a closure time of platelets isolated from the first subject before administration of meloxicam is more prolonged than a closure time of platelets isolated from an otherwise similar subject without platelet dysfunction.

In some embodiments, the pain is a moderate to severe pain. In some embodiments, the pain is an acute pain. In some embodiments, the meloxicam is present as nanocrystalline meloxicam. In some embodiments, the nanocrystalline meloxicam is in a colloidal dispersion. In some embodiments, meloxicam is administered to the first subject in an amount ranging from about 5 mg to about 180 mg. In some embodiments, meloxicam is administered to the first subject in an amount of about 30 mg. In some embodiments, meloxicam is administered to the first subject intravenously. In some embodiments, meloxicam is administered to the first subject intravenously over a course of about 5 seconds to about 60 seconds. In some embodiments, meloxicam is administered to the subject intravenously over a course of about 15 seconds.

In some embodiments, the first subject is a subject who will be subjected to a surgical procedure. In some embodiments, meloxicam is administered prior to start of a surgical procedure. In some embodiments, the first subject is not administered a non-steroidal anti-inflammatory drug, in combination with meloxicam. In some embodiments, the first subject is not administered a COX-1 inhibitor drug, in combination with meloxicam. In some embodiments, the first subject is administered acetaminophen, gabapentin, an opioid or a combination thereof, in combination with meloxicam. In some embodiments, the methods further comprise administering meloxicam about every 18 hours to about every 24 hours after a first administration of meloxicam.

In some embodiments, the clotting time of blood isolated from the first subject before administration of meloxicam is prolonged by about 1% to about 1000%, compared to the clotting time of blood isolated from the otherwise similar subject without platelet dysfunction. In some embodiments, the clotting time of blood isolated from the first subject before administration of meloxicam is prolonged by about 1%, about 5%, about 10%, about 20%, about 30%, about 40%, about 50%, about 60%, about 70%, about 80%, about 90%, about 100%, about 200%, about 300%, about 400%, about 500%, about 600%, about 700%, about 800%, about 900% or about 1000%, compared to the clotting time of blood isolated from the otherwise similar subject without platelet dysfunction.

In some embodiments, the closure time of platelets isolated from the first subject before administration of meloxicam is prolonged by about 1% to about 1000%, compared to that of the closure time of platelets isolated from the otherwise similar subject without platelet dysfunction. In some embodiments, the closure time of platelets isolated from the first subject before administration of meloxicam is prolonged by about 1%, about 5%, about 10%, about 20%, about 30%, about 40%, about 50%, about 60%, about 70%, about 80%, about 90%, about 100%, about 200%, about 300%, about 400%, about 500%, about 600%, about 700%, about 800%, about 900% or about 1000%, compared to the closure time of platelets isolated from the otherwise similar subject without platelet dysfunction.

In some embodiments, the closure time of platelets isolated from the first subject after administration of meloxicam is comparable to a closure time of platelets isolated from the first subject before administration of meloxicam. In some embodiments, the closure time of platelets isolated from the first subject after administration of meloxicam is comparable to a closure time of platelets isolated from a second subject, wherein the second subject has the platelet dysfunction, and wherein the second subject is not administered meloxicam.

In some embodiments, the clotting time of blood isolated from the first subject after administration of meloxicam is comparable to a clotting time of blood isolated from the first subject before administration of meloxicam. In some embodiments, the clotting time of blood isolated from the first subject after administration of meloxicam is comparable to a clotting time of blood isolated from a second subject, wherein the second subject has the platelet dysfunction, and wherein the second subject is not administered meloxicam.

In some embodiments, the closure time of platelets isolated from the first subject after administration of meloxicam is at least about 10% to about 100% less than a closure time of platelets isolated from a second subject, wherein the second subject has the platelet dysfunction, and wherein the second subject is administered 15 mg/mL of ketorolac. In some embodiments, the closure time of platelets isolated from the first subject after administration of meloxicam ranges from about 40% to about 50%, less than a closure time of platelets isolated from a second subject, wherein the second subject has the platelet dysfunction, and wherein the second subject is administered 15 mg/mL of ketorolac. In some embodiments, the closure time of platelets isolated from the first subject after administration of meloxicam is about 44% less than a closure time of platelets isolated from a second subject, wherein the second subject has the platelet dysfunction, and wherein the second subject is administered 15 mg/mL of ketorolac.

In some embodiments, the clotting time of blood isolated from the first subject after administration of meloxicam is at least about 5% to about 100% less than a clotting time of blood isolated from a second subject, wherein the second subject has the platelet dysfunction, and wherein the second subject is administered 15 mg/mL of ketorolac. In some embodiments, the clotting time of blood isolated from the first subject after administration of meloxicam is at least about 5%, about 10%, about 20%, about 30%, about 40%, about 50%, about 60%, about 70%, about 80%, about 90%, or about 100%, less than a clotting time of blood isolated from a second subject, wherein the second subject has the platelet dysfunction, and wherein the second subject is administered 15 mg/mL of ketorolac. In some embodiments, the meloxicam is administered in a volume of about 1 mL.

### BRIEF DESCRIPTION OF FIGURES

FIG. 1 shows the dose response analysis based on data with CADP (FIG. 1A) and CEPI (FIG. 1B) (final analysis set [8 subjects]). CADP, collagen/adenosine diphosphate; CEPI, collagen/epinephrine.

FIG. 2 shows the gender effect analysis and depicts the difference (female−male) in closure time (seconds) using CADP reagent (FIG. 2A) and CEPI reagent (FIG. 2B).

### DETAILED DESCRIPTION

### Definitions

It is to be understood that the terminology used herein is for the purpose of describing particular embodiments only and is not intended to be limiting.

Unless defined otherwise, all technical and scientific terms used herein have the same meaning as commonly understood to one of ordinary skill in the art to which the present application belongs. Although any methods and materials similar or equivalent to those described herein can be used in the practice or testing of the present application, representative methods and materials are herein described.

Following long-standing patent law convention, the terms “a”, “an”, and “the” refer to “one or more” when used in this application, including the claims. Thus, for example, reference to “a carrier” includes mixtures of one or more carriers, two or more carriers, and the like and reference to “the method” includes reference to equivalent steps and/or methods known to those skilled in the art, and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in the present specification and attached claims are approximations that can vary depending upon the desired properties sought to be obtained by the present application. Generally the term “about”, as used herein in references to a measurable value such as an amount of weight, time, dose, etc. is meant to encompass values within an acceptable degree of variability in the art. In some embodiments, degree of variability is based on FDA guidelines.

As used herein, “meloxicam” refers to 4-hydroxy-2-methyl-N-(5-methyl-2-thiazolyl)-2H-1,2-benzothiazine-3-carboxamide-1,1-dioxide, which has the structure as depicted below. The molecular weight is 351.4. Its molecular formula is C₁₄H₁₃N₃O₄S₂.

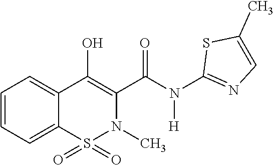 [SMILES: Cc1cnc(NC(=O)C2=C(O)c3ccccc3S(=O)(=O)N2C)s1]

As used herein, the term “bolus dose” refers to a discrete amount of a medication or a drug, e.g., meloxicam, which is given within a specific time. The specific time over which the bolus dose is administered (also referred to herein as the infusion rate) may be any suitable time which provides rapid onset of action (i.e., pain relief) and which does not cause significant injection site pain, such as a significant burning sensation. In some embodiments, the infusion time may be about 1 minute or less, e.g., about 30 seconds or about 15 seconds.

As used herein, “treatment” is an approach for obtaining beneficial or desired clinical results. For purposes of this invention, beneficial or desired clinical results include, but are not limited to, one or more of the following: improvement in any aspect of the pain including lessening severity, alleviation of one or more symptoms associated with pain including any aspect of pain (such as resting pain and/or mechanically-induced pain, shortening duration of pain, and/or reduction of pain sensitivity or sensation), reducing the incidence of, managing, ameliorating, preventing, and/or the delaying the development or progression of pain.

The term “effective amount” or “therapeutically effective amount” refers to the amount of an agent that is sufficient to achieve an outcome, for example, to effect beneficial or desired results. The therapeutically effective amount may vary depending upon one or more of: the subject and disease condition being treated, the weight and age of the subject, the severity of the disease condition, the manner of administration and the like.

The term “concurrent” or “concurrently” refers to administering two or more drugs close in time to each other. In some embodiments, the two or more concurrently administered drugs are administered within 1 hour, within about 30 min, within about 15 minutes, within about 10 minutes, within about 5 minutes of each other. In some embodiments, the two or more concurrently administered drugs are administered simultaneously.

As used herein, the term “subject” includes humans and other animals. Typically, the subject is a human. For example, the subject may be an adult, a teenager, a child (2 years to 14 years of age), an infant (1 month to 24 months), or a neonate (up to 1 month). In some embodiments, the adults are seniors about 65 years or older, or about 60 years or older. In some embodiments, the subject is a pregnant woman or a woman intending to become pregnant. In other aspects, subject is not a human; for example a non-human primate; for example, a baboon, a chimpanzee, a gorilla, or a macaque. In certain aspects, the subject may be a pet, such as a dog or cat.

As used herein, an “otherwise similar” subject refers to a subject that has the same or similar age and physiological characteristics.

As used herein, a closure time of platelets isolated from a first subject is referred to as being “comparable” to a closure time of platelets isolated from a second subject, when the closure time of the first subject is different from the closure time of the second subject by not more than 10%, for example, about 1%, about 2%, about 3%, about 4%, about 5%, about 6%, about 7%, about 8%, about 9%, including all values and subranges that lie therebetween.

### Therapeutic Use

While oral administration of meloxicam is approved for treating inflammation (e.g., osteoarthritis and rheumatoid arthritis), currently available oral formulations of meloxicam are known to have a slow onset of action due to poor solubility of meloxicam. The slow onset of action of oral meloxicam has rendered meloxicam not appropriate for acute pain management (e.g., mild to moderate pain and/or moderate to severe pain).

The inventors discovered that an intravenous formulation of meloxicam may be administered prior to a surgical procedure and/or in combination with additional therapeutic agents to provide a rapid onset of action of meloxicam that is critical for treatment of acute pain, such as surgical pain. Meloxicam nanocrystals significantly improves the solubility of the meloxicam, allowing for higher concentrations of meloxicam to be administered intravenously compared to an otherwise similar formulation in which meloxicam is not prepared as nanocrystals. Specifically, the inventors found that a meloxicam dose of about 5 mg to about 200 mg can provide a rapid onset of action of meloxicam while being efficacious and safe for the treatment of acute pain (e.g., mild to moderate pain and/or moderate to severe pain). In contrast to other intravenous NSAIDs such as ibuprofen and ketorolac, meloxicam nanocrystals can be safely administered intravenously without causing injection site pain. In addition, the inventors found that a bolus dose given over about 60 seconds (e.g., about 1 to about 60 seconds, about 1 to about 30 seconds, about 15 to about 30 seconds, etc.) was safe and effective for the treatment of pain. In some embodiments, the administration of intravenous meloxicam provided pain relief to the subject within about 15 minutes to within about 24 hours, for example, within about 15 minutes, within about 30 minutes, within about 1 hour, within about 2 hours, within about 3 hours, within about 4 hours, within about 5 hours, within about 6 hours, within about 12 hours, within about 18 hours, within about 24 hours, inclusive of all the values and subranges therebetween.

In one embodiment, the methods disclosed herein comprise administering to the subject a dose of meloxicam intravenously, wherein the meloxicam is at a dose of about 30 mg. In some embodiments, the methods disclosed herein comprise administering to the subject a dose of meloxicam intravenously, wherein the meloxicam is at a concentration of about 30 mg/mL. In one embodiment, the intravenous dose is a bolus dose. In some embodiments, the drug doses may be adjusted for subjects based on the surgery that is to be performed on the subject, age of the subject and the clinical condition of the subject. In some embodiments, the meloxicam is administered intramuscularly.

In one embodiment, the meloxicam is in a form of meloxicam nanocrystals. In another embodiment, meloxicam nanocrystals are formed using Alkermes NanoCrystal™ technology. See U.S. Pat. No. 8,512,727 which is hereby incorporated by reference in its entirety for all purposes.

In one embodiment of the method as disclosed herein, the IV dose (including a bolus dose) of meloxicam is administered to the subject over the course of about 1 to about 60 seconds, including all values and subranges therebetween. That is, the IV dose of meloxicam may be administered to a subject in about 1 second, about 2 seconds, about 3 seconds, about 4 seconds, about 5 seconds, about 6 seconds, about 7 seconds, about 8 seconds, about 9 seconds, about 10 seconds, about 11 seconds, about 12 seconds, about 13 seconds, about 14 seconds, about 15 seconds, about 16 seconds, about 17 seconds, about 18 seconds, about 19 seconds, about 20 seconds, about 21 seconds, about 22 seconds, about 23 seconds, about 24 seconds, about 25 seconds, about 26 seconds, about 27 seconds, about 28 seconds, about 29 seconds, about 30 seconds, about 31 seconds, about 32 seconds, about 33 seconds, about 34 seconds, about 35 seconds, about 36 seconds, about 37 seconds, about 38 seconds, about 39 seconds, about 40 seconds, about 41 seconds, about 42 seconds, about 43 seconds, about 44 seconds, about 45 seconds, about 46 seconds, about 47 seconds, about 48 seconds, about 49 seconds, about 50 seconds, about 51 seconds, about 52 seconds, about 53 seconds, about 54 seconds, about 55 seconds, about 56 seconds, about 57 seconds, about 58 seconds, about 59 seconds, or about 60 seconds, or any ranges between these values.

For example, in some embodiments, the IV dose (including a bolus dose) of meloxicam is administered to the subject over the course of about 5 to about 45 seconds. In other embodiments, the IV dose of meloxicam is administered to the subject over the course of about 10 to about 40 seconds. In still other embodiments, the IV dose of meloxicam is administered to the subject over the course of about 15 to about 35 seconds. In some embodiments, the IV dose of meloxicam is administered to the subject over the course of about 10 to about 30 seconds. In certain embodiments, the IV dose of meloxicam is administered to the subject over the course of about 15 to about 30 seconds. In one embodiment, the IV dose of meloxicam is administered to the subject over about 15 seconds.

The infusion rates of the present disclosure are significantly quicker than the FDA-approved infusion time of CALDOLOR® (an intravenous formulation of the NSAID ibuprofen), which requires at least 30 minutes. See CALDOLOR® Prescribing Information. Similarly, the infusion rates of the present disclosure are also significantly faster than infusion rates for OFIRMEV® (an intravenous formulation of acetaminophen), which requires a 15 minute infusion rate. See OFIRMEV® Prescribing Information. Whereas intravenous formulations of ibuprofen and acetaminophen cause injection site pain when administered at a rate that is faster than 15 minutes and 30 minutes, respectively, the present formulations were surprisingly discovered not to cause such injection site pain when administered in a IV dose (including a bolus dose).

Further, the inventors discovered that an injection of meloxicam within seconds, according to the methods disclosed herein, achieves fast onset of analgesics which is critical for management of acute pain, such as post-surgical pain. For example, in one embodiment, the dose of meloxicam administered intravenously to a subject can provide pain relief within about 10 minutes. This rapid onset of pain relief provided by the methods of the present disclosure is a substantial improvement from available intravenous NSAIDs, such as ketorolac which can take up to 30 minutes for the onset of pain relief. See Ketorolac Tromethamine Injection Prescribing Information. In other embodiments, the dose of meloxicam can be administered intravenously to a subject prior to surgery and advantageously treat post-surgical pain. In further or alternative embodiments, meloxicam can be administered in combination with other therapeutic agents to provide pain relief.

Moreover, unlike the previously reported NSAID injections which resulted in high injection site pain adverse effects (e.g., 16%-24% reported), the inventors found that the injection methods for administration of meloxicam disclosed herein is safe and efficacious, as only 2% of subjects receiving a dose of intravenous meloxicam reported injection site pain.

In one embodiment of the methods disclosed herein, the dose of meloxicam is in the range of from about 1 mg to about 250 mg, inclusive of all values and subranges therebetween. That is, the dose of meloxicam may be about 1 mg, about 2 mg, about 3 mg, about 4 mg, about 5 mg, about 6 mg, about 7 mg, about 8 mg, about 9 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, 225 mg, about 230 mg, 235 mg, about 240 mg, 245 mg, or about 250 mg, or any ranges between these values.

In one embodiment, the dose of meloxicam is in the range of from about 5 mg to about 200 mg. In some embodiments, the dose of meloxicam is in the range of from about 15 mg to about 180 mg. In some embodiments, the dose of meloxicam is in the range of from about 15 mg to about 100 mg. In other embodiments, the dose of meloxicam is in the range of from about 15 mg to about 80 mg. In some embodiments, the dose of meloxicam is in the range of from about 20 mg to about 70 mg. In some embodiments, the dose of meloxicam is in the range of from about 30 mg to about 60 mg. In some embodiments, the dose of meloxicam is about 30 mg. In another embodiment, the dose of meloxicam is about 60 mg.

In some embodiments, the intravenous meloxicam is formulated at a concentration of from about 10 mg/mL to about 50 mg/mL, e.g., about 10 mg/mL, about 15 mg/mL, about 20 mg/mL, about 25 mg/mL, about 30 mg/mL, about 35 mg/mL, about 40 mg/mL, about 45 mg/mL, about 50 mg/mL, about 55 mg/mL, and about 60 mg/mL, inclusive of all values and subranges therebetween. In particular embodiments, the intravenous meloxicam is formulated at a concentration of about 30 mg/mL.

In some embodiments, the dose of meloxicam as disclosed herein is administered once a day, twice a day, three times a day, every other day, or at a frequency deemed appropriate by a physician. In one embodiment, the dose of meloxicam is administered once a day intravenously. In some embodiments, meloxicam is administered every 18-26 hours until the subject is no longer in need thereof. As used herein, a “subject is no longer in need thereof” when the pain has subsided or the subject is discharged from the hospital. In some embodiments, meloxicam is administered intravenously once every 12 hours, once every 18 hours, once every 24 hours, once every 36 hours, once every 48 hours or at a frequency deemed appropriate by a physician. In particular embodiments, meloxicam is administered once every 24 hours.

In some embodiments, the dose of meloxicam as disclosed herein can be administered once a day for 1 day, 2 days, 3 days, 4 days, 5 days, 6 days, 7 days, 8 days, 9 days, 10 days, 11 days, 12 days, 13 days, 14 days, or at a duration and frequency deemed appropriate by a physician.

In one embodiment, a single dose, including a bolus dose, as disclosed herein can provide a rapid treatment which lasts for about 12 hours to about 48 hours. In one embodiment, a single dose as disclosed herein can provide a rapid treatment which lasts for about 24 hours. The ability for the presently disclosed meloxicam formulation to provide treatment lasting about 24 hours is a significant improvement over previously approved NSAID IV treatments, such as CALDOLOR® which requires infusion every 6 hours. See CALDOLOR® Prescribing Information.

In any of the methods disclosed herein, meloxicam can be administered for treatment of pain or for pain management. In one embodiment, meloxicam can be administered for the treatment or management of moderate to severe pain. In one embodiment, meloxicam can be administered for the treatment or management of mild to moderate pain. In one embodiment, the pain management is for a human subject. In one embodiment, the human subject is an adult.

### Formulations

In one embodiment, the dose for an IV injection or an IV infusion disclosed herein can comprise one or more pharmaceutically acceptable excipients or carriers known to one skilled in the art.

In one embodiment, a pharmaceutically acceptable excipient for the dose for an IV injection or an IV infusion can include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, sodium deoxycholate (deoxycholic acid), starch tragacanth, sucrose or xanthan gum.

In one embodiment, the dose of meloxicam disclosed herein for injection or infusion can be formulated in liquid carriers such as, water, dextrose in water, glucose in water, vegetable oil, alcohol, polyethylene glycol, propylene glycol, or glycerin. In one embodiment, the dose of meloxicam disclosed herein for injection is formulated in sterile water.

In one embodiment, the dose of meloxicam is in a form of aqueous dispersion.

In one embodiment of the method as disclosed herein, the dose of meloxicam is present in a volume of from about 0.5 mL to about 4 mL, inclusive of all values and subranges therebetween. That is, the IV dose (including a bolus dose) of meloxicam is present in a volume of about 0.5 mL, about 0.6 mL, about 0.7 mL, about 0.8 mL, about 0.9 mL, about 1.0 mL, about 1.1 mL, about 1.2 mL, about 1.3 mL, about 1.4 mL, about 1.5 mL, about 1.6 mL, about 1.7 mL, about 1.8 mL, about 1.9 mL, about 2.0 mL, about 2.1 mL, about 2.2 mL, about 2.3 mL, about 2.4 mL, about 2.5 mL, about 2.6 mL, about 2.7 mL, about 2.8 mL, about 2.9 mL, about 3.0 mL, about 3.1 mL, about 3.2 mL, about 3.3 mL, about 3.4 mL, about 3.5 mL, about 3.6 mL, about 3.7 mL, about 3.8 mL, about 3.9 mL, or about 4.0 mL, or any ranges between these values. In another embodiment, the dose of meloxicam is present in a volume of about 1 mL.

In one embodiment of the method as disclosed herein, the dose of meloxicam is present at a concentration of about 30 mg/mL. That is, the dose of meloxicam can be present at a concentration between about 28.5 mg/mL and about 31.5 mg/mL or any subranges between the two values. In one embodiment, the dose of meloxicam can be present at a concentration of about 22 mg/mL, about 23 mg/mL, about 24 mg/mL, about 25 mg/mL, about 26 mg/mL, about 27 mg/mL, about 28 mg/mL, about 29 mg/mL, about 30 mg/mL, about 31 mg/mL, about 32 mg/mL, about 33 mg/mL, about 34 mg/mL, about 35 mg/mL, about 36 mg/mL, about 37 mg/mL, or about 38 mg/mL, inclusive of all values and subranges therebetween. In one embodiment, the dose of meloxicam as disclosed herein can be a bolus dose.

In one embodiment, the dose of meloxicam is present at a concentration of about 30 mg/mL as a single use vial.

As previously noted, meloxicam has poor water solubility, which is one of the main reasons oral administration is not suitable for treatment of acute pain. Further, due to meloxicam's poor water solubility, it is challenging to provide an injectable formulation that is sufficiently concentrated so that the formulation can be injected to subjects in seconds in order to achieve rapid onset of pain relief without causing injection site pain. However, the inventors were able to increase the meloxicam concentration to 30 mg/mL by using meloxicam nanocrystals. This is a 20% increase in the concentration as compared to an otherwise similar formulation in which meloxicam is not prepared as nanocrystals, which is substantial considering meloxicam is poorly water soluble. The concentration of meloxicam as disclosed herein is critical to providing an IV dose and achieving rapid onset of pain relief without causing injection site pain. At concentrations of meloxicam which are higher than those disclosed herein, the drugs may crystalize out of solution, which may interfere with the injectability and/or syringeability of the formulation. At concentrations of meloxicam which are lower than those disclosed herein, the larger volumes of carrier preclude administration within the time ranges specified herein, and thereby cannot achieve rapid onset of pain relief.

In one embodiment, the dose of meloxicam as disclosed herein is used with dilution. In one embodiment, the dose of meloxicam as disclosed herein is used without dilution. In one embodiment, the 30 mg/mL dose of meloxicam is used without dilution. In one embodiment, the 30 mg/mL dose of meloxicam is not added to an IV solution or an IV fluid bag. That is, the 30 mg/mL dose of meloxicam as disclosed herein is administered to a subject in need thereof as 30 mg/mL.

### Pharmacokinetics

In one embodiment, a single 30 mg/mL bolus dose provides an average blood plasma Cₘₐₓ within about 80% to about 125% of the range of from about 4000 ng/mL to about 11000 ng/mL in a subject after intravenous administration of 30 mg of meloxicam, inclusive of all values and subranges therebetween. That is, a single 30 mg/mL bolus dose can provide plasma Cₘₐₓ of about 3000 ng/mL, about 3100 ng/mL, about 3200 ng/mL, about 3300 ng/mL, about 3400 ng/mL, about 3500 ng/mL, about 3600 ng/mL, about 3700 ng/mL, about 3800 ng/mL, about 3900 ng/mL, about 4000 ng/mL, about 4100 ng/mL, about 4200 ng/mL, about 4300 ng/mL, about 4400 ng/mL, about 4500 ng/mL, about 4600 ng/mL, about 4700 ng/mL, about 4800 ng/mL, about 4900 ng/mL, about 5000 ng/mL, about 5100 ng/mL, about 5200 ng/mL, about 5300 ng/mL, about 5400 ng/mL, about 5500 ng/mL, about 5600 ng/mL, about 5700 ng/mL, about 5800 ng/mL, about 5900 ng/mL, about 6000 ng/mL, about 6100 ng/mL, about 6200 ng/mL, about 6300 ng/mL, about 6400 ng/mL, about 6500 ng/mL, about 6600 ng/mL, about 6700 ng/mL, about 6800 ng/mL, about 6900 ng/mL, about 7000 ng/mL, about 7100 ng/mL, about 7200 ng/mL, about 7300 ng/mL, about 7400 ng/mL, about 7500 ng/mL, about 7600 ng/mL, about 7700 ng/mL, about 7800 ng/mL, about 7900 ng/mL, about 8000 ng/mL, about 8100 ng/mL, about 8200 ng/mL, about 8300 ng/mL, about 8400 ng/mL, about 8500 ng/mL, about 8600 ng/mL, about 8700 ng/mL, about 8800 ng/mL, about 8900 ng/mL, about 9000 ng/mL, about 9100 ng/mL, about 9200 ng/mL, about 9300 ng/mL, about 9400 ng/mL, about 9500 ng/mL, about 9600 ng/mL, about 9700 ng/mL, about 9800 ng/mL, about 9900 ng/mL, about 10000 ng/mL, about 10100 ng/mL, about 10200 ng/mL, about 10300 ng/mL, about 10400 ng/mL, about 10500 ng/mL, about 10600 ng/mL, about 10700 ng/mL, about 10800 ng/mL, about 10900 ng/mL, about 11000 ng/mL, about 11100 ng/mL, about 11200 ng/mL, about 11300 ng/mL, about 11400 ng/mL, about 11500 ng/mL, about 11600 ng/mL, about 11700 ng/mL, about 11800 ng/mL, about 11900 ng/mL, about 12000 ng/mL, about 12100 ng/mL, about 12200 ng/mL, about 12300 ng/mL, about 12400 ng/mL, about 12500 ng/mL, about 12600 ng/mL, about 12700 ng/mL, about 12800 ng/mL, about 12900 ng/mL, about 13000 ng/mL, about 13100 ng/mL, about 13200 ng/mL, about 13300 ng/mL, about 13400 ng/mL, and about 13500 ng/mL, or any values or ranges between above values, in a subject.

In one embodiment, a 1 mL bolus of 30 mg/mL provides an average plasma Cₘₐₓ within about 80% to about 125% of the range of from about 5642.9±1009.0 ng/mL in a subject after intravenous administration of intravenous meloxicam, inclusive of all values and subranges therebetween. In one embodiment, a single 30 mg/mL bolus dose provides an average plasma Cₘₐₓ within the range of from about 3707.1 ng/mL to about 8314.9 ng/mL in a subject after intravenous administration of intravenous meloxicam, inclusive of all values and subranges therebetween. In one embodiment, a single 30 mg/mL bolus dose provides an average plasma Cₘₐₓ within about 80% to about 125% of the range of from about 4000 ng/mL to about 7000 ng/mL in a subject after intravenous administration of 30 mg of meloxicam, inclusive of all values and subranges therebetween. In one embodiment, a single 30 mg/mL bolus dose provides an average plasma Cₘₐₓ within about 80% to about 125% of the range of from about 4600 ng/mL to about 6700 ng/mL in a subject after intravenous administration of intravenous meloxicam, inclusive of all values and subranges therebetween. In one embodiment, a single 30 mg/mL bolus dose provides an average plasma Cₘₐₓ within about 80% to about 125% of the range of from about 5000 ng/mL to about 6000 ng/mL in a subject after intravenous administration of 30 mg of meloxicam, inclusive of all values and subranges therebetween.

In one embodiment, a single 30 mg/mL bolus dose provides plasma Cₘₐₓ within about 80% to about 125% of the range of from about 7972.5±2579.0 ng/mL in a subject after intravenous administration of 30 mg of meloxicam, inclusive of all values and subranges therebetween. In one embodiment, a single 30 mg/mL bolus dose provides plasma Cₘₐₓ within the range of from about 4,312.1 ng/mL to about 13,190.5 ng/mL in a subject after intravenous administration of 30 mg of meloxicam, inclusive of all values and subranges therebetween. In one embodiment, a single 30 mg/mL bolus dose provides an average plasma Cₘₐₓ within about 80% to about 125% of the range of from about 5000 ng/mL to about 11000 ng/mL in a subject after intravenous administration of 30 mg of meloxicam, inclusive of all values and subranges therebetween. In one embodiment, a single 30 mg/mL bolus dose provides an average plasma Cₘₐₓ within about 80% to about 125% of the range of from about 5500 ng/mL to about 10500 ng/mL in a subject after intravenous administration of 30 mg of meloxicam, inclusive of all values and subranges therebetween. In one embodiment, a single 30 mg/mL bolus dose provides an average plasma Cₘₐₓ within about 80% to about 125% of the range of from about 7000 ng/mL to about 9000 ng/mL in a subject after intravenous administration of intravenous meloxicam, inclusive of all value sand subranges therebetween.

In one embodiment, a repeat dose (e.g., administered once daily) of a 30 mg/mL bolus dose provides plasma Cₘₐₓ (e.g., a steady state Cₘₐₓ) within about 80% to about 125% of the range of from about 10632.5±4729.8 ng/mL in a subject after intravenous administration of 30 mg of meloxicam, inclusive of all values and subranges therebetween. In one embodiment, a repeat dose of a 30 mg/mL bolus dose provides plasma Cₘₐₓ within the range of from about 4,722.2 ng/mL to about 19,202.9 ng/mL in a subject after intravenous administration of 30 mg of meloxicam, inclusive of all values and subranges therebetween. That is, a repeat dose of 30 mg/mL bolus dose can provide plasma Cₘₐₓ of about 4500 ng/mL, about 4600 ng/mL, about 4700 ng/mL, about 4800 ng/mL, about 4900 ng/mL, about 5000 ng/mL, about 5100 ng/mL, about 5200 ng/mL, about 5300 ng/mL, about 5400 ng/mL, about 5500 ng/mL, about 5600 ng/mL, about 5700 ng/mL, about 5800 ng/mL, about 5900 ng/mL, about 6000 ng/mL, about 6100 ng/mL, about 6200 ng/mL, about 6300 ng/mL, about 6400 ng/mL, about 6500 ng/mL, about 6600 ng/mL, about 6700 ng/mL, about 6800 ng/mL, about 6900 ng/mL, about 7000 ng/mL, about 7100 ng/mL, about 7200 ng/mL, about 7300 ng/mL, about 7400 ng/mL, about 7500 ng/mL, about 7600 ng/mL, about 7700 ng/mL, about 7800 ng/mL, about 7900 ng/mL, about 8000 ng/mL, about 8100 ng/mL, about 8200 ng/mL, about 8300 ng/mL, about 8400 ng/mL, about 8500 ng/mL, about 8600 ng/mL, about 8700 ng/mL, about 8800 ng/mL, about 8900 ng/mL, about 9000 ng/mL, about 9100 ng/mL, about 9200 ng/mL, about 9300 ng/mL, about 9400 ng/mL, about 9500 ng/mL, about 9600 ng/mL, about 9700 ng/mL, about 9800 ng/mL, about 9900 ng/mL, about 10000 ng/mL, about 10100 ng/mL, about 10200 ng/mL, about 10300 ng/mL, about 10400 ng/mL, about 10500 ng/mL, about 10600 ng/mL, about 10700 ng/mL, about 10800 ng/mL, about 10900 ng/mL, about 11000 ng/mL, about 11100 ng/mL, about 11200 ng/mL, about 11300 ng/mL, about 11400 ng/mL, about 11500 ng/mL, about 11600 ng/mL, about 11700 ng/mL, about 11800 ng/mL, about 11900 ng/mL, about 12000 ng/mL, about 12100 ng/mL, about 12200 ng/mL, about 12300 ng/mL, about 12400 ng/mL, about 12500 ng/mL, about 12600 ng/mL, about 12700 ng/mL, about 12800 ng/mL, about 12900 ng/mL, about 13000 ng/mL, about 13100 ng/mL, about 13200 ng/mL, about 13300 ng/mL, about 13400 ng/mL, about 13500 ng/mL, about 13600 ng/mL, about 13700 ng/mL, about 13800 ng/mL, about 13900 ng/mL, about 14000 ng/mL, about 14100 ng/mL, about 14200 ng/mL, about 14300 ng/mL, about 14400 ng/mL, about 14500 ng/mL, about 14600 ng/mL, about 14700 ng/mL, about 14800 ng/mL, about 14900 ng/mL, about 15000 ng/mL, about 15100 ng/mL, about 15200 ng/mL, about 15300 ng/mL, about 15400 ng/mL, about 15500 ng/mL, about 15600 ng/mL, about 15700 ng/mL, about 15800 ng/mL, about 15900 ng/mL, about 16000 ng/mL, about 16100 ng/mL, about 16200 ng/mL, about 16300 ng/mL, about 16400 ng/mL, about 16500 ng/mL, about 16600 ng/mL, about 16700 ng/mL, about 16800 ng/mL, about 16900 ng/mL, about 17000 ng/mL, about 17100 ng/mL, about 17200 ng/mL, about 17300 ng/mL, about 17400 ng/mL, about 17500 ng/mL, about 17600 ng/mL, about 17700 ng/mL, about 17800 ng/mL, about 17900 ng/mL, about 18000 ng/mL, about 18100 ng/mL, about 18200 ng/mL, about 18300 ng/mL, about 18400 ng/mL, about 18500 ng/mL, about 18600 ng/mL, about 18700 ng/mL, about 18800 ng/mL, about 18900 ng/mL, about 19000 ng/mL, about 19100 ng/mL, about 19200 ng/mL, about 19300 ng/mL, about 19400 ng/mL, about 19500 ng/mL, about 19600 ng/mL, about 19700 ng/mL, about 19800 ng/mL, about 19900 ng/mL, or about 12000 ng/mL, or any values or ranges between above values, in a subject.

In one embodiment, a repeat dose of a 30 mg/mL bolus dose provides plasma Cₘₐₓ within about 80% to about 125% of the range of from about 5000 ng/mL to about 20000 ng/mL in a subject after intravenous administration of 30 mg of meloxicam, inclusive of all values and subranges therebetween. In one embodiment, a repeat dose of 30 mg/mL bolus dose provides an average plasma Cₘₐₓ within about 80% to about 125% of the range of from about 7000 ng/mL to about 18000 ng/mL in a subject after intravenous administration of 30 mg of meloxicam, inclusive of all values and subranges therebetween. In one embodiment, a repeat dose of 30 mg/mL bolus dose provides an average plasma Cₘₐₓ within m about 80% to about 125% of the range of from about 8000 ng/mL to about 13000 ng/mL in a subject after intravenous administration of 30 mg of meloxicam, inclusive of all values and subranges therebetween.

In one embodiment, a single 30 mg/mL bolus dose provides an average plasma AUC_{inf} within about 80% to about 125% of the range of from about 55,000 ng*hr/mL to about 190,000 ng*hr/mL in a subject after intravenous administration of 30 mg of meloxicam, inclusive of all values and subranges therebetween. That is, a single 30 mg/mL bolus dose can provide an average plasma AUC_{inf} of about 40,000 ng*hr/mL, about 45,000 ng*hr/mL, about 50,000 ng*hr/mL, about 55,000 ng*hr/mL, about 60,000 ng*hr/mL, about 65,000 ng*hr/mL, about 70,000 ng*hr/mL, about 75,000 ng*hr/mL, about 80,000 ng*hr/mL, about 85,000 ng*hr/mL, about 90,000 ng*hr/mL, about 95,000 ng*hr/mL, about 100,000 ng*hr/mL, about 105,000 ng*hr/mL, about 110,000 ng*hr/mL, about 115,000 ng*hr/mL, about 120,000 ng*hr/mL, about 125,000 ng*hr/mL, about 130,000 ng*hr/mL, about 135,000 ng*hr/mL, about 140,000 ng*hr/mL, about 145,000 ng*hr/mL, about 150,000 ng*hr/mL, about 155,000 ng*hr/mL, about 160,000 ng*hr/mL, about 165,000 ng*hr/mL, about 170,000 ng*hr/mL, about 175,000 ng*hr/mL, about 180,000 ng*hr/mL, about 185,000 ng*hr/mL, about 190,000 ng*hr/mL, about 195,000 ng*hr/mL, about 200,000 ng*hr/mL, about 205,000 ng*hr/mL, about 210,000 ng*hr/mL, about 215,000 ng*hr/mL, about 220,000 ng*hr/mL, about 225,000 ng*hr/mL, about 230,000 ng*hr/mL, about 235,000 ng*hr/mL, and about 240,000 ng*hr/mL, or any values or ranges between above values, in a subject.

In one embodiment, a single 30 mg/mL bolus dose provides an average plasma AUC_{inf} of within about 80% to about 125% of the range of from about 107508.7±34443.0 ng*hr/mL in a subject after intravenous administration of 30 mg of meloxicam, inclusive of all values and subranges therebetween. In one embodiment, a single 30 mg/mL bolus dose provides an average plasma AUC_{inf} within the range of from about 58,452.6 ng*hr/mL to about 177,440.0 ng*hr/mL in a subject after intravenous administration of 30 mg of meloxicam, inclusive of all values and subranges therebetween. In one embodiment, a single 30 mg/mL bolus dose provides an average plasma AUC_{inf} within about 80% to about 125% of the range of from about 121437.6±64505.6 ng*hr/mL in a subject after intravenous administration of 30 mg of meloxicam, inclusive of all values and subranges therebetween. In one embodiment, a single 30 mg/mL bolus dose provides an average plasma AUC_{inf} within the range of from about 45,545.6 ng*hr/mL to about 232,429.0 ng*hr/mL in a subject after intravenous administration of 30 mg of meloxicam, inclusive of all values and subranges therebetween. In one embodiment, a single 30 mg/mL bolus dose provides an average plasma AUC_{inf} of about 70,000 ng*hr/mL to about 190,000 ng*hr/mL in a subject after intravenous administration of 30 mg of meloxicam, inclusive of all values and subranges therebetween. In one embodiment, a single 30 mg/mL bolus dose provides an average plasma AUC_{inf} within the range of from about 80% to about 125% of about 70,000 ng*hr/mL to about 140,000 ng*hr/mL in a subject after intravenous administration of 30 mg of meloxicam, inclusive of all values and subranges therebetween. In one embodiment, a single 30 mg/mL bolus dose provides an average plasma AUC_{inf} within the range of from about 80% to about 125% of about 75,000 ng*hr/mL to about 130,000 ng*hr/mL in a subject after intravenous administration of 30 mg of meloxicam, inclusive of all values and subranges therebetween. In one embodiment, a single 30 mg/mL bolus dose provides an average plasma AUC_{inf} within the range of from about 80% to about 125% of about 85,000 ng*hr/mL to about 120,000 ng*hr/mL in a subject after intravenous administration of 30 mg of meloxicam, inclusive of all values and subranges therebetween. In one embodiment, a single 30 mg/mL bolus dose provides an average plasma AUC_{inf} within the range of from about 80% to about 125% of about 55,000 ng*hr/mL to about 190,000 ng*hr/mL in a subject after intravenous administration of 30 mg of meloxicam, inclusive of all values and subranges therebetween. In one embodiment, a single 30 mg/mL bolus dose provides an average plasma AUC_{inf} within about 80% to about 125% of the range of from about 80,000 ng*hr/mL to about 160,000 ng*hr/mL in a subject after intravenous administration of 30 mg of meloxicam, inclusive of all values and subranges therebetween. In one embodiment, a single 30 mg/mL bolus dose provides an average plasma AUC_{inf} of about within about 80% to about 125% the range of from about 100,000 ng*hr/mL to about 140,000 ng*hr/mL in a subject after intravenous administration of 30 mg of meloxicam, inclusive of all values and subranges therebetween.

In one embodiment, a repeat dose (e.g., more than one dose) of a 30 mg/mL bolus dose provides plasma AUC_{inf} within about 80% to about 125% of the range of from about 297,771.6±241,604.01 ng*hr/mL in a subject after intravenous administration of 30 mg of meloxicam, inclusive of all values and subranges therebetween. In one embodiment, a repeat dose of a 30 mg/mL bolus dose provides plasma AUC_{inf} within the range of from about 44,934.1 ng*hr/mL to about 674,219.5 ng*hr/mL in a subject after intravenous administration of 30 mg of meloxicam, inclusive of all values and subranges therebetween. In one embodiment, a repeat dose of a 30 mg/mL bolus dose provides an average plasma AUC_{inf} within about 80% to about 125% of the range of from about 55,000 ng*hr/mL to about 540,000 ng*hr/mL in a subject after intravenous administration of 30 mg of meloxicam, inclusive of all values and subranges therebetween. In one embodiment, a repeat dose of a 30 mg/mL bolus dose provides an average plasma AUC_{inf} within about 80% to about 125% of the range of from about 80,000 ng*hr/mL to about 500,000 ng*hr/mL in a subject after intravenous administration of 30 mg of meloxicam, inclusive of all values and subranges therebetween. In one embodiment, a repeat dose of a 30 mg/mL bolus dose provides an average plasma AUC_{inf} of about within about 80% to about 125% the range of from about 100,000 ng*hr/mL to about 450,000 ng*hr/mL in a subject after intravenous administration of 30 mg of meloxicam, inclusive of all values and subranges therebetween. In one embodiment, a repeat dose of a 30 mg/mL bolus dose provides an average plasma AUC_{inf} of about within about 80% to about 125% the range of from about 150,000 ng*hr/mL to about 400,000 ng*hr/mL in a subject after intravenous administration of 30 mg of meloxicam, inclusive of all values and subranges therebetween. In one embodiment, a repeat dose of a 30 mg/mL bolus dose provides an average plasma AUC_{inf} of about within about 80% to about 125% the range of from about 200,000 ng*hr/mL to about 350,000 ng*hr/mL in a subject after intravenous administration of 30 mg of meloxicam, inclusive of all values and subranges therebetween. In one embodiment, a repeat dose of a 30 mg/mL bolus dose provides an average plasma AUC_{inf} of about within about 80% to about 125% the range of from about 250,000 ng*hr/mL to about 325,000 ng*hr/mL in a subject after intravenous administration of 30 mg of meloxicam, inclusive of all values and subranges therebetween.

In one embodiment, steady state can be achieved upon repeat dose of a 30 mg bolus dose administered intravenously once daily for 7 days.

In one embodiment, a single 30 mg/mL bolus IV dose provides an average plasma Tₘₐₓ of about 0.05 h to about 0.20 h in a subject after intravenous administration of 30 mg of meloxicam, inclusive of all values and subranges therebetween. That is, a single 30 mg/mL bolus IV injection can provide an average plasma Tₘₐₓ of about 0.05 h, about 0.06 h, about 0.07 h, about 0.08 h, about 0.09 h, about 0.10 h, about 0.11 h, about 0.12 h, about 0.13 h, about 0.14 h, about 0.15 h, about 0.16 h, about 0.17 h, about 0.18 h, about 0.19 h, or about 0.20 h, or any values or ranges between above values, in a subject.

In one embodiment, a single 30 mg/mL bolus dose provides an average plasma Tₘₐₓ of about 0.08 h to about 0.16 h in a subject after intravenous administration of 30 mg of meloxicam, inclusive of all values and subranges therebetween. In one embodiment, a single 30 mg/mL bolus dose provides an average plasma Tₘₐₓ of about 0.10 h to about 0.14 h in a subject after intravenous administration of 30 mg of meloxicam, inclusive of all values and subranges therebetween.

An orally administered meloxicam has a prolonged absorption, with a mean plasma Tₘₐₓ of about 5-7 hours following administration. The methods as disclosed herein provides significantly faster Tₘₐₓ, e.g., about 0.08 h to about 0.16 h following administration, which is indicative of rapid onset and fast absorption.

In one embodiment, the method as disclosed herein can provide meloxicam peak analgesic effect within about 30 minutes to about 60 minutes. That is, the administration of 30 mg/mL bolus IV injection of meloxicam can provide peak analgesic effect in about 30 minutes, about 35 minutes, about 40 minutes, about 45 minutes, about 50 minutes, about 55 minutes, or about 60 minutes, or any values or ranges between above values. In one embodiment, the administration of 30 mg/mL bolus IV injection of meloxicam can provide peak analgesic effect in about 40 minutes.

Not only is the meloxicam administration as disclosed herein provide a fast onset of pain relief, it also reaches peak analgesic effect sooner than other known IV NSAIDs (Ketorolac can take 1 to 2 hours for maximum effect) and has a longer therapeutic window of at least about 24 hours (Ketorolac's duration of analgesic effect is 4 to 6 hours). See Ketorolac Tromethamine Injection Prescribing Information.

In one embodiment, a 1 mL bolus of 30 mg/mL of meloxicam provides an average plasma concentration in the range of from about 80% to about 125% of 4160±1020 ng/mL of meloxicam in a subject at about 30 minutes after intravenous administration, inclusive of all values and subranges therebetween. In one embodiment, a single 30 mg/mL bolus dose of meloxicam provides an average plasma concentration in the range of from about 2512 ng/mL to about 6475 ng/mL of meloxicam in a subject at about 30 minutes after intravenous administration, inclusive of all values and subranges therebetween. In some embodiments, a single 30 mg/mL bolus dose of meloxicam provides an average plasma concentration in the range of from about 3000 ng/mL to about 6000 ng/mL of meloxicam in a subject at about 30 minutes after intravenous administration, inclusive of all values and subranges therebetween. In other embodiments, a single 30 mg/mL bolus dose of meloxicam provides an average plasma concentration in the range of from about 3500 ng/mL to about 5500 ng/mL of meloxicam in a subject at about 30 minutes after intravenous administration, inclusive of all values and subranges therebetween. In some embodiments, a single 30 mg/mL bolus dose of meloxicam provides an average plasma concentration in the range of from about 3500 ng/mL to about 5000 ng/mL of meloxicam in a subject at about 30 minutes after intravenous administration, inclusive of all values and subranges therebetween.

In one embodiment, a 1 mL bolus of 30 mg/mL of meloxicam provides an average plasma concentration in the range of from about 80% to about 125% of 3590±708 ng/mL of meloxicam in a subject at about 60 minutes after intravenous administration, inclusive of all values and subranges therebetween. In one embodiment, a single 30 mg/mL bolus dose of meloxicam provides an average plasma concentration in the range of from about 2305 ng/mL to about 5373 ng/mL of meloxicam in a subject at about 60 minutes after intravenous administration, inclusive of all values and subranges therebetween. In some embodiments, a single 30 mg/mL bolus dose of meloxicam provides an average plasma concentration in the range of from about 2500 ng/mL to about 5000 ng/mL of meloxicam in a subject at about 60 minutes after intravenous administration, inclusive of all values and subranges therebetween. In other embodiments, a single 30 mg/mL bolus dose of meloxicam provides an average plasma concentration in the range of from about 2750 ng/mL to about 4500 ng/mL of meloxicam in a subject at about 60 minutes after intravenous administration, inclusive of all values and subranges therebetween. In some embodiments, a single 30 mg/mL bolus dose of meloxicam provides an average plasma concentration in the range of from about 3000 ng/mL to about 4000 ng/mL of meloxicam in a subject at about 60 minutes after intravenous administration, inclusive of all values and subranges therebetween.

In one embodiment, a single 30 mg/mL bolus dose of meloxicam provides an average plasma concentration in the range of from about 80% to about 125% of about 2660±394 ng/mL of meloxicam in a subject at about 120 minutes after intravenous administration, inclusive of all values and subranges therebetween. In one embodiment, a single 30 mg/mL bolus dose of meloxicam provides an average plasma concentration in the range of from about 1812 ng/mL to about 3818 ng/mL of meloxicam in a subject at about 120 minutes after intravenous administration, inclusive of all values and subranges therebetween. In some embodiments, a single 30 mg/mL bolus dose of meloxicam provides an average plasma concentration in the range of from about 1900 ng/mL to about 3800 ng/mL of meloxicam in a subject at about 120 minutes after intravenous administration, inclusive of all values and subranges therebetween. In other embodiments, a single 30 mg/mL bolus dose of meloxicam provides an average plasma concentration in the range of from about 2100 ng/mL to about 3600 ng/mL of meloxicam in a subject at about 120 minutes after intravenous administration, inclusive of all values and subranges therebetween. In some embodiments, a single 30 mg/mL bolus dose of meloxicam provides an average plasma concentration in the range of from about 2200 ng/mL to about 3400 ng/mL of meloxicam in a subject at about 120 minutes after intravenous administration, inclusive of all values and subranges therebetween.

In one embodiment, a single 30 mg/mL bolus dose of meloxicam provides an average plasma concentration in the range of from about 80% to about 125% of about 2190±262 ng/mL of meloxicam in a subject at about 4 hours after intravenous administration, inclusive of all values and subranges therebetween. In one embodiment, a single 30 mg/mL bolus dose of meloxicam provides an average plasma concentration in the range of from about 1542 ng/mL to about 3065 ng/mL of meloxicam in a subject at about 4 hours after intravenous administration, inclusive of all values and subranges therebetween. In some embodiments, a single 30 mg/mL bolus dose of meloxicam provides an average plasma concentration in the range of from about 1600 ng/mL to about 3000 ng/mL of meloxicam in a subject at about 4 hours after intravenous administration, inclusive of all values and subranges therebetween. In other embodiments, a single 30 mg/mL bolus dose of meloxicam provides an average plasma concentration in the range of from about 1800 ng/mL to about 2800 ng/mL of meloxicam in a subject at about 4 hours after intravenous administration, inclusive of all values and subranges therebetween. In some embodiments, a single 30 mg/mL bolus dose of meloxicam provides an average plasma concentration in the range of from about 1900 ng/mL to about 2600 ng/mL of meloxicam in a subject at about 4 hours after intravenous administration, inclusive of all values and subranges therebetween.

### Platelet Dysfunction

The disclosure provides methods of treating pain in a subject in need thereof, comprising administering meloxicam to the subject. In some embodiments, the subject has a platelet dysfunction. As used herein, “platelet dysfunction” or “platelet disorder” refers to a disease, disorder or condition in a subject, in which platelet function is affected or impaired. Platelets function by reacting to bleeding from blood vessel injury by clumping, thereby initiating a blood clot. Without being bound by theory, it is thought that platelet dysfunction is associated with, promotes, or causes an increased risk of excessive bleeding due to injuries and/or spontaneous bleeding.

The function of platelets may be assessed using any one of the methods described here, or known in the art, such as those described in Paniccia et. al., Vascular Health and Risk Management, 2015:11 133-148, the contents of which are incorporated herein by reference in its entirety for all purposes. Non-limiting examples of methods that may be used to assess the function of platelets include complete blood count, prothrombin time [PT] and partial thromboplastin time [PTT], bleeding time, light transmission platelet aggregation, lumiaggregometry, impedance aggregometry on whole blood, platelet activation investigated by flow cytometry, PFA-100, VerifyNow System, and Multiplate Electrode Aggregometry [MEA].

In some embodiments, platelet dysfunction may be associated with, promoted by, or result from a change in the numbers of platelets in the subject, as compared to a healthy subject. As used herein, the term “healthy subject” refers to an otherwise similar subject not suffering from platelet dysfunction. In some embodiments, a normal platelet count in a healthy subject ranges from about 150,000 to about 450,000 platelets per microliter of blood. In some embodiments, the platelet dysfunction comprises an increase in platelet numbers in the subject as compared to a healthy subject. Non-limiting examples of conditions in which platelet dysfunction is characterized by an increase in platelet numbers include thrombocythemia or reactive thrombocytosis. In some embodiments, the subject has a platelet count of more than about 450,000 platelets per microliter of blood, for example, about 500,000 platelets per microliter of blood, about 550,000 platelets per microliter of blood, about 600,000 platelets per microliter of blood, about 650,000 platelets per microliter of blood, about 700,000 platelets per microliter of blood, about 750,000 platelets per microliter of blood, about 800,000 platelets per microliter of blood, about 850,000 platelets per microliter of blood, about 900,000 platelets per microliter of blood, about 950,000 platelets per microliter of blood, including all subranges and values that lie therebetween.

In some embodiments, the platelet dysfunction comprises a decrease in platelet numbers in the subject, as compared to a healthy subject, also called, thrombocytopenia. In some embodiments, the subject has a platelet count of less than about 150,000 platelets per microliter of blood, for example, about 125,000 platelets per microliter of blood, about 100,000 platelets per microliter of blood, about 75,000 platelets per microliter of blood, about 50,000 platelets per microliter of blood, about 25,000 platelets per microliter of blood, about 20,000 platelets per microliter of blood, about 15,000 platelets per microliter, about 10,000 platelets per microliter, or about 5,000 platelets per microliter, including any subranges or values that lie therebetween. In some embodiments, the subject has a platelet count of less than about 50,000 platelets per microliter. In some embodiments, the subject has a platelet count of less than about 10,000 per microliter of blood or less than about 25,000 platelets per microliter of blood. In some embodiments, thrombocytopenia is associated with pregnancy (gestational thrombocytopenia), administration of drugs that cause immune-mediated platelet destruction (such as, heparin, trimethoprim/sulfamethoxazole, quinine, and others described herein), administration of drugs that cause dose-dependent bone marrow suppression (such as, chemotherapeutic agents, ethanol, and others described herein), and systemic infection or immune disorders (such as, immune thrombocytopenia).

In some embodiments, platelet dysfunction may be associated with, promoted by, or result from a deficiency or change in the function of at least one platelet in the subject, as compared to a healthy subject.

In some embodiments, the platelet dysfunction is an inherited platelet dysfunction. Non-limiting examples of inherited platelet dysfunction include Von Willebrand disease, Glanzmann disease, Wiskott-Aldrich syndrome, Chédiak-Higashi syndrome, Bernard-Soulier syndrome, Gray platelet syndrome, Hermansky-Pudlak syndrome, Chediak-Higashi syndrome, Scott syndrome, Wiskott-Aldrich syndrome, X-linked thrombocytopenia, CAMT, thrombocytopenia-absent radius, Velocardialfacial syndrome, Mediterranean macrothrombocytopenia, May-Hegglin anomaly, Fechtner syndrome, Sebastian syndrome, Epstein syndrome and any one of the diseases described in D'Andrea et al, Blood Transfus. 2009 October; 7(4): 278-292, the contents of which are herein incorporated by reference it its entirety for all purposes.

In some embodiments, the platelet dysfunction is an acquired platelet dysfunction. In some embodiments, the acquired platelet dysfunction is associated with, promoted by, or caused by the administration of at least one blood thinning drug to the subject. In some embodiments, the at least one blood thinning drug is at least one anti-platelet drug. Non-limiting examples of anti-platelet drugs include aspirin, clopidogrel, prasugrel, ticagrelor, dipyridamole, triflusal, cangrelor, ticlopidine, cilostazol, vorapaxar, abciximab, eptifibatide, tirofiban, epoprostenol, thromboxane synthase inhibitors, thromboxane receptor antagonists such as tertroban, and ticlopidine.

In some embodiments, the at least one anti-platelet drug is a nonsteroidal anti-inflammatory drug. Non-limiting examples of nonsteroidal anti-inflammatory drugs include salicylates such as, Aspirin (acetylsalicylic acid), Diflunisal (Dolobid), Salicylic acid and its salts, Salsalate (Disalcid); Propionic acid derivatives, such as, Ibuprofen, Dexibuprofen, Naproxen, Fenoprofen, Ketoprofen, Dexketoprofen, Flurbiprofen, Oxaprozin, Loxoprofen; Acetic acid derivatives, such as, Indomethacin, Tolmetin, Sulindac, Etodolac, Ketorolac, Diclofenac, Aceclofenac, Nabumetone; Anthranilic acid derivatives (fenamates), such as, Mefenamic acid, Meclofenamic acid, Flufenamic acid, Tolfenamic acid; Selective COX-2 inhibitors (coxibs), such as, Celecoxib, Parecoxib, Lumiracoxib, Etoricoxib, Firocoxib; Sulfonanilides, such as, Nimesulide; and others such as, Clonixin, Licofelone, H-harpagide and Devil's Claw.

In some embodiments, the at least one blood thinning drug is at least one anti-coagulant. Non-limiting examples of anti-coagulants include coumarins such as warfarin, acenocoumarol, phenprocoumon, atromentin and phenindione; heparin and derivatives such as low molecular weight heparin; synthetic pentasaccharide inhibitors of factor Xa, such as fondaparinux, idraparinux, and idrabiotaparinux; directly acting oral anticoagulants such as, dabigatran, rivaroxaban, apixaban, edoxaban and betrixaban; direct thrombin inhibitors such as hirudin, lepirudin, bivalirudin, argatroban and dabigatran; antithrombin protein therapeutics, enoxaparin, batroxobin, hementin and vitamin E. Therefore, in some embodiments, the subject with platelet dysfunction has been or is being administered an anti-coagulant. In some embodiments, the subject with platelet dysfunction may suffer from a condition that requires the administration of an anti-coagulant. Anti-coagulant may be administered for any condition that is known in the art to be treated or alleviated by the anti-coagulant. Non-limiting examples of conditions for which one or more anti-coagulant is administered to the subject include orthopedic procedures, and thoracic procedures.

In some embodiments, the subject with platelet dysfunction has been or is being administered a blood thinning drug in combination with one or more medications that increases the anti-platelet effects of the blood thinning drug. Non-limiting examples of medications that increase the anti-platelet effects of a blood thinning drug include cytotoxic drugs or drugs associated with bone marrow suppression (e.g.: Leflunamide, Hydrochloroquine, Adalimumab, Infliximab, Etanercept, Sulfasalazine, Penicillamine, Gold, Methotrexate, Azathioprine, Mycophenolate); other anticoagulants or antiplatelet drugs; and drugs affecting the nervous system (e.g.: Selective serotonin reuptake inhibitors (SSRIs)).

In some embodiments, the platelet dysfunction is associated with, promoted by, or results from cirrhosis, multiple myeloma, kidney disease, systemic lupus erythematosus, a disorder of secretion and thromboxane synthesis or uremia. In some embodiments, the platelet dysfunction is associated with, promoted by, or results from a cardiopulmonary bypass procedure. Therefore, in some embodiments, the subject had previously undergone a cardiopulmonary bypass procedure.

In some embodiments, the platelet dysfunction, and/or resulting blood thinning, is associated with, promoted by, or results from the administration of chemotherapy, spinal dysplastic disorders and/or liver diseases. Therefore, in some embodiments, the subject with platelet dysfunction has been or is being administered chemotherapy. In some embodiments, the subject with platelet dysfunction has one or more spinal dysplastic disorders and/or one or more liver diseases. Non-limiting examples of liver diseases include diseases caused by viruses, such as hepatitis A, hepatitis B, and hepatitis C; diseases caused by drugs, poisons, or too much alcohol, such as fatty liver disease and cirrhosis; liver cancer; and inherited diseases, such as hemochromatosis and Wilson disease.

In some embodiments, the clotting time of blood isolated from the subject with platelet dysfunction (before administration of meloxicam) is more prolonged than the clotting time of blood isolated from an otherwise similar subject without platelet dysfunction. In some embodiments, the clotting time of blood isolated from the subject with platelet dysfunction (before administration of meloxicam) is prolonged by about 1% to about 1000%, including any value or subrange that lies therebetween, compared to the clotting time of blood isolated from an otherwise similar subject without platelet dysfunction. In some embodiments, the clotting time of blood isolated from the subject with platelet dysfunction (before administration of meloxicam) is prolonged by about 1%, about 5%, about 10%, about 20%, about 30%, about 40%, about 50%, about 60%, about 70%, about 80%, about 90%, about 100%, about 200%, about 300%, about 400%, about 500%, about 600%, about 700%, about 800%, about 900% or about 1000%, compared to the clotting time of blood isolated from an otherwise similar subject without platelet dysfunction.

In some embodiments, a closure time of platelets isolated from the subject with platelet dysfunction (before administration of meloxicam) is more prolonged than a closure time of platelets isolated from an otherwise similar subject without platelet dysfunction. In some embodiments, the closure time of platelets isolated from the subject with platelet dysfunction (before administration of meloxicam) is prolonged by about 1% to about 1000%, including any value or subrange that lies therebetween, compared to the closure time of platelets isolated from an otherwise similar subject without platelet dysfunction. In some embodiments, the closure time of platelets isolated from the subject with platelet dysfunction (before administration of meloxicam) is prolonged by about 1%, about 5%, about 10%, about 20%, about 30%, about 40%, about 50%, about 60%, about 70%, about 80%, about 90%, about 100%, about 200%, about 300%, about 400%, about 500%, about 600%, about 700%, about 800%, about 900% or about 1000%, compared to the closure time of platelets isolated from an otherwise similar subject without platelet dysfunction

In some embodiments, the administration of meloxicam does not exacerbate the platelet dysfunction of the subject. For instance, in some embodiments, the closure time of platelets isolated from the subject after administration of meloxicam is comparable to a closure time of platelets isolated from the subject before administration of meloxicam. In some embodiments, the clotting time of blood isolated from the subject after administration of meloxicam is comparable to a clotting time of blood isolated from the subject before administration of meloxicam.

In some embodiments, the closure time of platelets isolated from the first subject after administration of meloxicam is comparable to a closure time of platelets isolated from a second subject, wherein the second subject has the platelet dysfunction, and wherein the second subject is not administered meloxicam. In some embodiments, a clotting time of blood isolated from the first subject after administration of meloxicam is comparable to a clotting time of blood isolated from a second subject, wherein the second subject has the platelet dysfunction, and wherein the second subject is not administered meloxicam.

In some embodiments, the closure time of platelets isolated from a first subject having platelet dysfunction after administration of meloxicam is less than a closure time of platelets isolated from a second subject having the platelet dysfunction, wherein the second subject is administered ketorolac. In some embodiments, the closure time of platelets isolated from a first subject having platelet dysfunction after administration of meloxicam is at least about 10% to about 100% less than a closure time of platelets isolated from a second subject having the platelet dysfunction, wherein the second subject is administered ketorolac. In some embodiments, the closure time of platelets isolated from the first subject after administration of meloxicam is at least about 10%, about 20%, about 30%, about 40%, about 50%, about 60%, about 70%, about 80%, about 90%, or about 100%, including all values and subranges that lie therebetween, less than a closure time of platelets isolated from a second subject having the platelet dysfunction, wherein the second subject is administered ketorolac. Methods for comparing meloxicam and ketorolac on platelet dysfunction are described in the examples.

The concentration of ketorolac administered to the second subject is not limited, and may be in the range of about 1 mg/mL to about 50 mg/mL, for example, about 1 mg/mL, about 2.5 mg/mL, about 5 mg/mL, about 7.5 mg/mL, about 10 mg/mL, about 12.5 mg/mL, about 15 mg/mL, about 20 mg/mL, about 30 mg/mL, about 40 mg/mL, or about 50 mg/mL, including all values and subranges that lie therebetween. In some embodiments, the concentration of ketorolac administered to the second subject is about 15 mg/mL or about 30 mg/mL.

In some embodiments, the closure time of platelets isolated from the first subject after administration of meloxicam is about 40% to about 50%, for example, about 40%, about 41%, about 42%, about 43%, about 44%, about 45%, about 46%, about 47%, about 48%, about 49%, about 50%, including all values and subranges that lie therebetween, less than a closure time of platelets isolated from a second subject having the platelet dysfunction, wherein the second subject is administered ketorolac. In some embodiments, the closure time of platelets isolated from the first subject after administration of 30 mg/mL meloxicam is in the range of about 40% to about 45%, for example about 41%, about 42%, about 43%, about 44%, about 45%, inclusive of all subranges and values that lie therebetween, less than the closure time of platelets isolated from a second subject having the platelet dysfunction, wherein the second subject is administered 15 mg/mL ketorolac. In some embodiments, the closure time of platelets isolated from the first subject after administration of 30 mg/mL meloxicam is about 44% less than the closure time of platelets isolated from a second subject having the platelet dysfunction, wherein the second subject is administered 15 mg/mL ketorolac.

In some embodiments, the clotting time of blood isolated from a first subject having platelet dysfunction after administration of meloxicam is less than a clotting time of blood isolated from a second subject having the platelet dysfunction, wherein the second subject is administered ketorolac. In some embodiments, the clotting time of blood isolated from a first subject having platelet dysfunction after administration of meloxicam is at least about 5% to about 100%, including all values and subranges that lie therebetween, less than a clotting time of blood isolated from a second subject having the platelet dysfunction, wherein the second subject is administered ketorolac. In some embodiments, the clotting time of blood isolated from the first subject after administration of meloxicam is at least about 5%, about 10%, about 20%, about 30%, about 40%, about 50%, about 60%, about 70%, about 80%, about 90%, or about 100%, including all values and subranges that lie therebetween, less than a clotting time of blood isolated from a second subject having the platelet dysfunction, wherein the second subject is administered ketorolac.

### EXAMPLES

### Example 1: Assessment of the Effects of Meloxicam IV on Platelet Function

Introduction: Non-steroidal anti-inflammatory drugs (NSAIDs) are an integral part of the World Health Organization (WHO) Pain Ladder as a first line defense to prevent or treat pain. Without being bound by any theory, it is thought that a concern of NSAID use in the surgical setting is the risk of perioperative or postoperative bleeding complications caused by inhibition of cyclooxygenase (COX) activity and prostaglandin biosynthesis resulting in the loss of platelet adhesion. Therefore, platelet dysfunction and a high risk for bleeding events has been associated with reductions in thromboxane resulting from inhibition of cyclooxygenase-1 (COX-1) by nonselective NSAIDs. Although a lower risk of bleeding events is observed with the use of COX-2-selective NSAIDs, highly COX-2-selective NSAIDs are associated with an increased risk of cardiovascular events (e.g., thrombosis, myocardial infarction) relative to COX-1-selective agents. Meloxicam, an NSAID with a long duration of action has preferential, but not exclusive, inhibition of COX-2 and a more favorable gastrointestinal adverse event profile compared with nonselective NSAIDs. Oral meloxicam has demonstrated efficacy in the treatment of chronic pain (eg, rheumatoid arthritis, osteoarthritis). However, oral meloxicam is not indicated for the treatment of acute pain, primarily because it has poor solubility and slow absorption. Peak concentrations occur 2.5 to 7 hours after oral administration of a 15-mg dose and 9 to 11 hours after a 30-mg dose, resulting in a delayed onset of action.

Intravenous meloxicam (meloxicam IV) described herein utilizes a nanocrystal formulation of meloxicam which can be used for the management of moderate to severe pain alone or in combination with other analgesics. Meloxicam IV was evaluated in four phase 2 and three phase 3 postoperative studies in subjects with moderate to severe pain following hard-tissue or soft-tissue surgeries (see Christensen et al, J Clin Pharmacol. 2018; 58(5):593-605; Gottlieb I J et al. J Pain Res. 2018; 11:383-393; Pollak R, et al., Clin J Pain. 2018; 34(10):918-926; Bergese S et al., Postgrad Med. 2017; 129(suppl 1):57-58; Rechberger T et al., Anesth Analg. 2018; 128(6):1309-1318; and Singla N et al., Plast Reconstr Surg Glob Open. 2018; 6(6): e1846, the contents of each of which is herein incorporated by reference in their entireties for all purposes.

The objective of this study was to determine whether meloxicam IV affects platelet function in comparison to both negative (untreated) and positive (ketorolac-treated) controls when assessed by an ex vivo analysis of whole blood samples from healthy subjects. Each whole blood sample was aliquoted to allow analysis using the PFA-100 under negative control (untreated), positive control (2 therapeutic ketorolac concentrations), and meloxicam IV (1 therapeutic, 3 supratherapeutic concentrations) using both collagen with epinephrine (CEPI) and collagen with adenosine diphosphate (CADP) reagent cartridges. The PFA-100 device determines closure time by simulating platelet adhesion and aggregation following vascular injury. The final analysis set included data from 8 healthy subjects.

Methods:

Study design: This was an ex vivo study conducted at a single center in the United States. The study was conducted according to US Food and Drug Administration regulations governing clinical trials, Title 21 Code of Federal Regulations Parts 50, 54, 56, and 312; International Conference on Harmonisation Good Clinical Practice Guidelines, and other regulations as applicable. The study was reviewed and approved by the study site's institutional review board (Midlands Independent Review Board, Overland Park, Kans.), and all subjects provided written informed consent.

Key eligibility criteria: Healthy males or females (aged 18-40 years) who were non-tobacco users (i.e., never used or stopped using at least 6 months prior to screening visit) were eligible for enrollment. Subjects were excluded if they had taken any medications (prescription or over-the-counter) or supplements (e.g., vitamins) within 14 days prior to blood collection at screening; if they were females of childbearing potential using hormonal contraception; or if they had a history of anemia or thrombocytopenia, alcohol abuse (i.e., regularly drinks >4 units of alcohol per day), or prescription/illicit drug abuse within 5 years. Subjects were also not allowed to have received any investigational product within 30 days prior to screening or to have received meloxicam IV in previous clinical trials.

Study material preparation, blood collection, and sample processing: Meloxicam IV 30 mg/mL drug product (Recro Pharma, Inc., Malvern, Pa.; Batch No. 30004) and ketorolac injection 15 mg/mL (Red Rock Pharmacy, Salt Lake City, Utah; Batch No. 67-031-DK) were diluted with 5% dextrose in water within 2 hours prior to study use. The final solution concentrations were 0.33 μg/μL for meloxicam IV and 0.1667 μg/μL for ketorolac (Table 1). One preparation of diluted meloxicam and ketorolac solution was utilized for all treated samples for each individual subject.

TABLE 1
Timing and Sequence of Sample Analysis

| Testing | End | |
|---|---|---|
| Sequence | Concentration | PFA-100 Reagent Cartridge |
| 1^{a} | NA (untreated control) | CEPI |
| 2^{a} | NA (untreated control) | CADP |
| 3 | Meloxicam 5 μg/mL | CEPI |
| 4 | Meloxicam 5 μg/mL | CADP |
| 5 | Meloxicam 10 μg/mL | CEPI |
| 6 | Meloxicam 10 μg/mL | CADP |
| 7 | Meloxicam 15 μg/mL | CEPI |
| 8 | Meloxicam 15 μg/mL | CADP |
| 9 | Meloxicam 20 μg/mL | CEPI |
| 10 | Meloxicam 20 μg/mL | CADP |
| 11 | Ketorolac 2.5 μg/mL | CEPI |
| 12 | Ketorolac 2.5 μg/mL | CADP |
| 13 | Ketorolac 5 μg/mL | CEPI |
| 14 | Ketorolac 5 μg/mL | CADP |

^{a}If PFA-100 closure time for control samples with CEPI reagent (Test 1) was ≥150 seconds OR control samples with CADP reagent (Test 2) was ≥110 seconds, sample analysis was discontinued, and no further samples were processed from the subject's blood sample.
CEPI, collagen/epinephrine; CADP, collagen/adenosine diphosphate; NA, not applicable.

Study subjects (n=13) had approximately 20 mL of whole blood collected in tubes containing 3.2% (0.105 M) buffered sodium citrate (1 part anticoagulant to 9 parts blood). Each blood sample was aliquoted for untreated analysis (negative control), as well as for analysis of samples treated with ketorolac (positive control) and meloxicam IV. Meloxicam IV 0.33 μg/μL was added to whole blood aliquots to yield end concentrations of 5, 10, 15, and 20 μg/mL. This was designed to yield one sample that reflected approximate maximum plasma concentrations (Cₘₐₓ) following a 30 mg dose (i.e., 5 μg/mL), the anticipated therapeutic dose, and three samples with concentrations exceeding the exposure of the anticipated therapeutic dose. Similarly, ketorolac 0.1667 μg/μL was added to whole blood aliquots to yield end concentrations of 2.5 μg/mL and 5 μg/mL, which were designed to mimic peak concentrations following 15-mg and 30-mg IV ketorolac doses, respectively.

Assessment of platelet function: Platelet function was evaluated using the PFA-100 Platelet Function Analyzer (Siemens Healthcare Diagnostics, Deerfield, Ill.). The PFA-100 system is US Food and Drug Administration-approved to identify drug-induced platelet abnormalities under flow conditions that create a high shear, similar to flow through the blood vessel. Use of this device to measure the effect of drugs on platelet adhesion is well documented. The PFA-100 device determines a sample closure time by simulating the platelet adhesion and aggregation that occurs following vascular injury. Analysis was performed using both collagen with epinephrine (CEPI) and collagen with adenosine diphosphate (CADP) reagent cartridges. Each whole blood sample was aliquoted to allow analysis under negative control (untreated), positive control (2 ketorolac concentrations), and meloxicam IV (4 concentrations) using both the CADP and CEPI cartridges. Whole blood aliquots were treated according to the test condition and incubated for approximately 10 minutes prior to analysis in the PFA-100. All blood samples were analyzed within 2.5 hours of the time of collection. PFA-100 closure time results were reported for each test condition and reagent cartridge. Test results were evaluated for quality control based on a single repeat sample analysis within each subject, with an acceptance criterion of within 20% variance of the original result. Samples outside this range were excluded from the primary analysis.

Statistical analysis: Treatment effect on CT was analyzed using an analysis of variance (ANOVA). Treatment effect on closure time using PFA-100 was analyzed with an analysis of covariance model that had main effect of treatment and covariate of gender to assess treatment effect with (ie, the full model) and without (ie, the reduced model) controlling for covariates (treatment, gender, the interaction between treatment and gender). Treatment effect was analyzed twice: the first analysis (primary analysis) excluded samples that did not meet the quality-control criteria (n=8), whereas the second analysis (confirmatory analysis) included all samples (n=12; one subject excluded due to instrument malfunction). Pairwise comparisons were performed; nominal P-values were reported without controlling for multiplicity. Subgroup analysis by gender was also performed. All analyses were performed separately for each reagent.

Results: Whole blood samples were analyzed from 13 subjects (7 male, 6 female) using the PFA-100. The final analysis set included data from 8 subject samples, with 5 subject samples excluded. Reasons for exclusion included instrument malfunction in 1 subject, and out of range quality control sample results in 4 subjects.

CADP reagent analysis: Sample analysis using the collagen with adenosine diphosphate (CADP) reagent cartridge did not demonstrate a significant overall treatment effect on CT (p=0.5715). There were no statistically significant differences in closure time values between either the meloxicam IV- or ketorolac-treated samples and the untreated control samples (Table 2).

TABLE 2
Least Squares (LS) Mean Closure Times and Comparison by Treatment
Using CADP Reagent (final analysis set [8 subjects]).

| | | Ketorolac | | | Meloxicam IV | | |
|---|---|---|---|---|---|---|---|
| | Untreated | 2.5 | 5 | 5 | 10 | 15 | 20 |
| | Control | μg/mL | μg/mL | μg/mL | μg/mL | μg/mL | μg/mL |
| LS mean (SE) closure | 74.54 | 79.41 | 87.95 | 75.41 | 74.91 | 76.66 | 74.91 |
| time, seconds | (5.31) | (5.31) | (5.66) | (5.31) | (5.31) | (5.31) | (5.31) |
| P value vs untreated | | .5179 | .0907 | .9074 | .9602 | .7776 | .9602 |
| control | | | | | | | |
| P value vs 2.5 μg/mL | | | .2765 | .5955 | .5505 | .7148 | .5505 |
| ketorolac | | | | | | | |
| P value vs 5 μg/mL | | | | .1130 | .0997 | .1524 | .0997 |
| ketorolac | | | | | | | |

CADP, collagen/adcnosine diphosphate; SE, standard error.

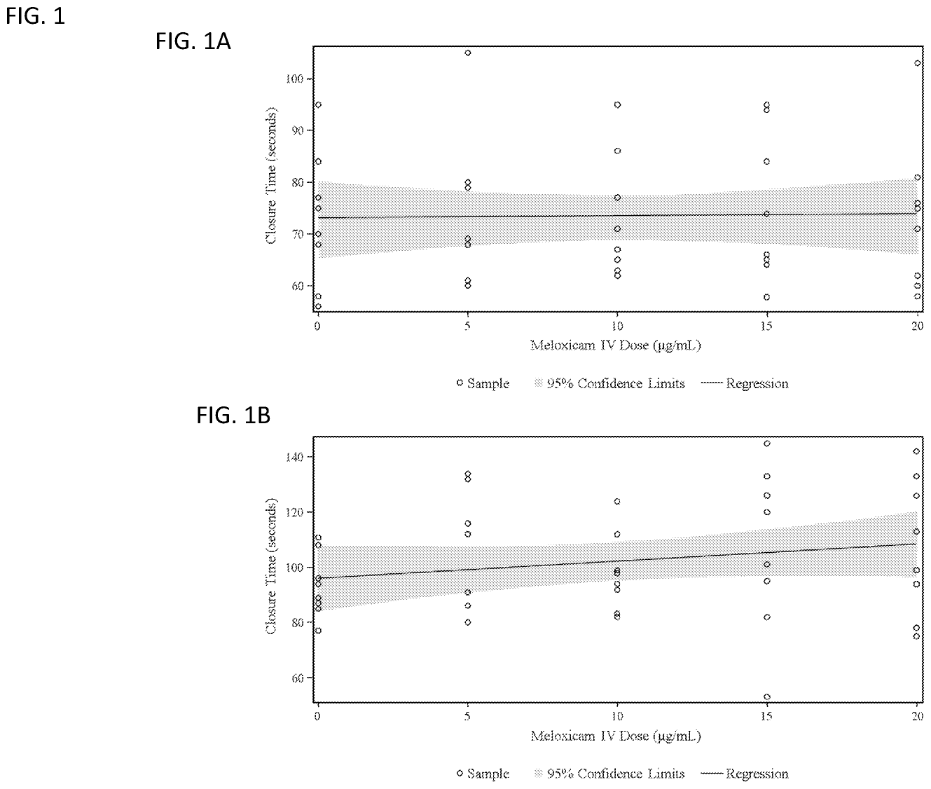

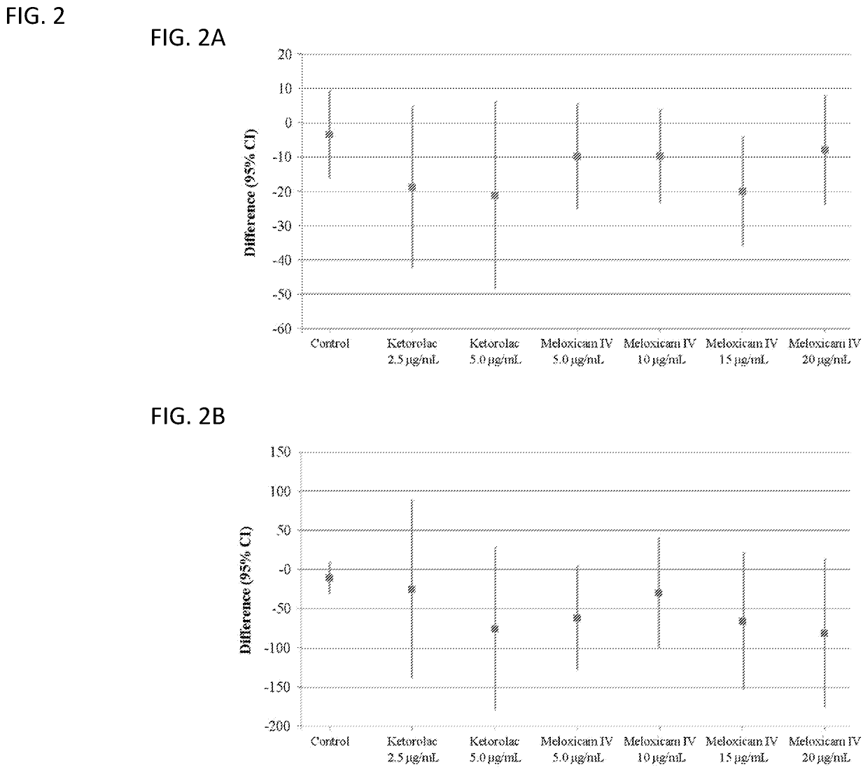

Mean closure times (standard error) for ketorolac 2.5- and 5-μg/mL samples and the meloxicam IV 5-, 10-, 15-, and 20-μg/mL samples were 79.41 (5.31), 87.95 (5.66), 75.41 (5.31), 74.91 (5.31), 76.66 (5.31), and 74.91 (5.31) seconds, respectively, compared with 74.54 (5.31) seconds for the untreated control. There were no statistically significant differences in closure time between any of the meloxicam IV-treated samples (i.e., 5, 10, 15, or 20 μg/mL) and either ketorolac-treated samples or between the two ketorolac-treated samples. A dose-response analysis showed no trend toward changes in closure time with increasing doses of meloxicam IV (FIG. 1A). There were also no significant differences between males and females for closure time in the CADP reagent analysis for any of the ketorolac or meloxicam IV concentrations (FIG. 2A). Results were generally similar in the confirmatory analysis when samples from all 12 subjects (excluding 1 sample with instrument malfunction) were included, with the exception that the ketorolac 5-μg/mL sample had a significantly longer closure time compared to the untreated control sample (P=0.0162) and to the meloxicam IV 10-μg/mL sample (P=0.0253) (Table 3).

TABLE 3
Least Squares (LS) Mean Closure Times and Comparison by Treatment Using CADP
Reagent (all eligible subjects without instrument errors [12 subjects]^{a}).

| | | Ketorolac | | | Meloxicam IV | | |
|---|---|---|---|---|---|---|---|
| | Untreated | 2.5 | 5 | 5 | 10 | 15 | 20 |
| | Control | μg/mL | μg/mL | μg/mL | μg/mL | μg/mL | μg/mL |
| LS mean (SE) closure | 71.42 | 82.08 | 86.36 | 75.42 | 72.50 | 77.75 | 76.25 |
| time, seconds | (4.18) | (4.18) | (4.37) | (4.18) | (448) | (4.18) | (4.18) |
| P value vs untreated | | .0761 | .0162 | .5014 | .8553 | .2884 | .4170 |
| control | | | | | | | |
| P value vs 2.5 μg/mL | | | .4822 | .2640 | .1102 | .4666 | .3279 |
| ketorolac | | | | | | | |
| P value vs 5 μg/mL | | | | .0752 | .0253 | .1596 | .0096 |
| ketorolac | | | | | | | |

^{a}Excludes one subject due to instalment malfunction.
CADP, collagen/adenosine diphosphate; SE, standard error.

CEPI reagent cartridge analysis: In the collagen with epinephrine (CEPI) reagent analysis, a significant treatment effect was observed for changes in CT (p=0.0441). When compared to untreated control, the ketorolac treated CT values were significantly prolonged in both the 2.5 μg/mL (p=0.0003) and 5 μg/mL (p=0.0257) concentrations using the CEPI reagent. In the meloxicam IV treated samples, no significant difference was observed in CT vs. untreated control at any of the evaluated concentrations using the CEPI reagent; p=0.6252, 0.8406, 0.5580, and 0.5400 for the 5, 10, 15, and 20 μg/mL concentrations respectively. When the meloxicam IV CT results were compared with the ketorolac IV results, all meloxicam IV concentrations had a significantly shorter CT compared to the 2.5 μg/mL ketorolac concentration (p<0.005), and numerically shorter CTs were observed compared to the 5 μg/mL ketorolac concentration, though only statistically significant at the 10 μg/mL meloxicam IV concentration (p=0.0408 to 0.0974). (Table 4).

TABLE 4
Least Squares (LS) Mean Closure Times and Comparison by Treatment
Using CEPI Reagent (final analysis set [8 subjects]).

| | | Ketorolac | | | Meloxicam IV | | |
|---|---|---|---|---|---|---|---|
| | Untreated | 2.5 | 5 | 5 | 10 | 15 | 20 |
| | Control | μg/mL | μg/mL | μg/mL | μg/mL | μg/mL | μg/mL |
| LS mean (SE) closure | 90.50 | 180.87 | 143.38 | 101.75 | 95.13 | 104.00 | 104.63 |
| time, seconds | (16.54) | (16.54) | (16.54) | (16.54) | (16.54) | (16.54) | (16.54) |
| P value vs untreated | | .0003 | .0257 | .6252 | .8406 | .5580 | .5400 |
| control | | | | | | | |
| P value vs 2.5 μg/mL | | | .1084 | .0012 | .0005 | .0017 | .0018 |
| ketorolac | | | | | | | |
| P value vs 5 μg/mL | | | | .0757 | .0408 | .0923 | .0974 |
| ketorolac | | | | | | | |

CEPI, collagen/epinephrine: SE, standard error.

The dose-response analysis observed a small trend of increased closure time with increasing doses of meloxicam IV (FIG. 1B). However, there were no statistically significant differences between any of the meloxicam IV-treated closure time values. There were also no significant differences between males and females for closure time for either ketorolac or meloxicam in the CEPI reagent analysis (FIG. 2B), with the exception that mean closure time was significantly greater in males than in females in the meloxicam 15-μg/mL concentration (89.9 vs 69.8 seconds; P=0.0180).

The overall results were generally similar in the confirmatory analysis when samples from all 12 subjects (excluding 1 sample with instrument malfunction) were included. Both ketorolac samples (2.5- and 5-μg/mL concentrations) demonstrated statistically significant longer closure times compared with the untreated control. Across the meloxicam IV samples (5-, 10-, 15-, and 20-μg/mL concentrations), none was associated with a statistically significant increase in closure time compared with untreated control (Table 5).

TABLE 5
Least Squares (LS) Mean Closure Times and Comparison by Treatment Using CEPI
Reagent (all eligible subjects without instrument errors [12 subjects]).

| | | Ketorolac | | | Meloxicam IV | | |
|---|---|---|---|---|---|---|---|
| | Untreated | 2.5 | 5 | 5 | 10 | 15 | 20 |
| | Control | μg/mL | μg/mL | μg/mL | μg/mL | μg/mL | μg/mL |
| LS Mean (SE) closure | 99.17 | 193.17 | 191.58 | 122.58 | 115.33 | 126.83 | 142.92 |
| time, seconds | (18.30) | (18.30) | (18.30) | (18.30) | (18.30) | (18.30) | (18.30) |
| P value vs untreated | | .0005 | .0007 | .3688 | .5343 | .2889 | .0956 |
| control | | | | | | | |
| P value vs 2.5 μg/mL | | | .9514 | .0082 | .0037 | .0127 | .0564 |
| ketorolac | | | | | | | |
| P value vs 5 μg/mL | | | | .0096 | .0044 | .0148 | .0644 |
| ketorolac | | | | | | | |

^{a}Excludes 1 subject due to instrument malfunction.
CEPI, collagen/epinephrine; SE, standard error.

Conclusion: Table 6 summarizes the COX selectivity of common NSAIDs as expressed by the ratio of the NSAID concentration that inhibited 80% of the activity (IC₈₀) of COX-2 to the IC₈₀ of COX-1. Agents range from relatively selective for COX-1 (eg, ketorolac) to those that are more selective for COX-2 (eg, meloxicam, celecoxib). Without being bound by a theory, it is thought that these differential effects on platelets have clinical significance, with nonselective NSAIDs being associated with a greater effect on platelet function and bleeding time compared with COX-2-selective NSAIDs, which do not inhibit thromboxane A₂.

TABLE 6
COX Selectivity of Common NSAIDs Based on the Ratio
of Concentrations Needed to Inhibit 80% of the Activity (IC₈₀)
of COX-2 to the IC₈₀ of COX-1 (see Warner 1999, the
contents of which was incorporated herein by reference
in its entirety for all purposes).

| | Agent | COX-2/COX-1 IC₈₀ ratio |
|---|---|---|
| Greater COX-1 selectivity | Ketorolac | 294 |
| | Aspirin | 3.8 |
| | Naproxen | 3 |
| | Ibuprofen | 2.6 |
| | Diclofenac | 0.23 |
| | Celecoxib | 0.11 |
| | Meloxicam | 0.091 |
| Greater COX-2 selectivity | Rofecoxib | <0.05 |

COX, cyclooxygenase; IC, inhibitory concentration; NSAID, nonsteroidal anti-inflammatory drug.

In the current study, there was no significant prolongation in closure time in meloxicam IV-treated whole blood samples, either at concentrations reflecting therapeutic levels or at supratherapeutic exposure levels, compared with untreated control when assessed by either the CADP or CEPI assay. In contrast, whole blood samples treated with therapeutic concentrations of ketorolac showed significant prolongations in closure time compared with untreated controls in the CEPI analysis. There were significant differences between meloxicam and ketorolac in the CEPI analysis at several drug concentrations. The differential effects in the CADP and CEPI analyses are consistent with the rationales of the two assays. CADP cartridges are primarily affected by thrombocytopathies with a lower sensitivity to aspirin effects, while CEPI cartridges have a high sensitivity to aspirin-induced platelet abnormalities. Overall, these data suggest that meloxicam IV has a lower risk than ketorolac for platelet dysfunction—related events.

The results demonstrating that ketorolac has a significant effect on platelet function are consistent with previous studies. In several studies in healthy volunteers, therapeutic doses of ketorolac (0.4 mg/kg) caused a significant inhibition of epinephrine-, adenosine-, and collagen-induced platelet aggregation and also prolonged bleeding times. Further, in studies evaluating the effect of ketorolac on platelet function in subjects undergoing surgical procedures, some, but not all, studies found that intravenous ketorolac was associated with inhibition of platelet aggregation and prolonged bleeding time.

In this study, a single repeat sample analysis from each subject was performed to assess quality control, with an acceptance criterion of within 20% variance of the original result. Four subjects were excluded from the CADP and CEPI analyses because repeat sample analysis for each subject had a greater than 20% variance from their original result. When samples from all 12 subjects were included, the overall results were generally similar in the confirmatory analyses for both the CADP and CEPI cartridges.

In sum, no significant prolongation in CT was observed in meloxicam IV treated whole blood samples at concentrations reflecting therapeutic and supratherapeutic exposures compared with untreated controls. In contrast, significant prolongations in CT were observed in ketorolac treated samples compared with control. These results suggest a potential clinical benefit of meloxicam IV over ketorolac with regard to a decreased risk of platelet dysfunction. These studies suggest that the administration of meloxicam to subjects with platelet dysfunction would not exacerbate the platelet dysfunction of the subject. Therefore, the administration of meloxicam to treat pain to subjects with platelet dysfunction, as per the methods disclosed herein, provides superior and unexpected advantages over the administration of the standard-of-care pain treatment medications.

What is claimed is:

1. A method of treating pain in a first subject in need thereof, comprising administering meloxicam to the first subject, wherein the first subject has a platelet dysfunction.

2. The method of claim 1, wherein the platelet dysfunction is an inherited platelet dysfunction.

3. The method of claim 2, wherein the inherited platelet dysfunction is Von Willebrand disease, Glanzmann disease, Wiskott-Aldrich syndrome, Chédiak-Higashi syndrome, or Bernard-Soulier syndrome.

4. The method of claim 1, wherein the platelet dysfunction is an acquired platelet dysfunction.

5. The method of claim 4, wherein the acquired platelet dysfunction is caused by an administration of at least one blood thinning drug to the first subject.

6. The method of claim 5, wherein the at least one blood thinning drug is at least one anti-platelet drug.

7. The method of claim 6, wherein the at least one anti-platelet drug is aspirin, clopidogrel, dipyridamole or ticlopidine.

8. The method of claim 6, wherein the at least one anti-platelet drug is a nonsteroidal anti-inflammatory drug.

9. The method of claim 5, wherein the at least one blood thinning drug is at least one anti-coagulant.

10. The method of claim 9, wherein the at least one anti-coagulant is warfarin, enoxaparin, heparin, dabigatran, apixaban, betrixaban or rivaroxaban.

11. The method of claims 1-10, wherein the first subject has at least one disease or condition that affects platelet function.

12. The method of claim 11, wherein the at least one disease or condition that affects platelet function is cirrhosis, multiple myeloma, kidney disease, systemic lupus erythematosus, a disorder of secretion and thromboxane synthesis or uremia.

13. The method of claims 1-12, wherein the first subject was previously subjected to a cardiopulmonary bypass procedure.

14. The method of claims 1-13, wherein a clotting time of blood isolated from the first subject before administration of meloxicam is more prolonged than a clotting time of blood isolated from an otherwise similar subject without platelet dysfunction.

15. The method of claims 1-14, wherein a closure time of platelets isolated from the first subject before administration of meloxicam is more prolonged than a closure time of platelets isolated from an otherwise similar subject without platelet dysfunction.

16. The method of claims 1-15, wherein the pain is a moderate to severe pain.

17. The method of claims 1-16, wherein the pain is an acute pain.

18. The method of claims 1-17, wherein the meloxicam is present as nanocrystalline meloxicam.

19. The method of claims 1-18, wherein the nanocrystalline meloxicam is in a colloidal dispersion.

20. The method of claims 1-19, wherein meloxicam is administered to the first subject in an amount ranging from about 5 mg to about 180 mg.

21. The method of claims 1-20, wherein meloxicam is administered to the first subject in an amount of about 30 mg.

22. The method of claims 1-21, wherein meloxicam is administered to the first subject intravenously.

23. The method of claims 1-22, wherein meloxicam is administered to the first subject intravenously over a course of about 5 seconds to about 60 seconds.

24. The method of claims 1-23, wherein meloxicam is administered to the subject intravenously over a course of about 15 seconds.

25. The method of claims 1-24, wherein the first subject is a subject who will be subjected to a surgical procedure.

26. The method of claim 25, wherein meloxicam is administered prior to start of a surgical procedure.

27. The method of claims 1-26, wherein the first subject is not administered a non-steroidal anti-inflammatory drug, in combination with meloxicam.

28. The method of claims 1-27, wherein the first subject is not administered a COX-1 inhibitor drug, in combination with meloxicam.

29. The method of claims 1-28, wherein the first subject is administered acetaminophen, gabapentin, an opioid or a combination thereof, in combination with meloxicam.

30. The method of claims 1-29, further comprising administering meloxicam about every 18 hours to about every 24 hours after a first administration of meloxicam.

31. The method of claim 14, wherein the clotting time of blood isolated from the first subject before administration of meloxicam is prolonged by about 1% to about 1000%, compared to the clotting time of blood isolated from the otherwise similar subject without platelet dysfunction.

32. The method of claim 31, wherein the clotting time of blood isolated from the first subject before administration of meloxicam is prolonged by about 1%, about 5%, about 10%, about 20%, about 30%, about 40%, about 50%, about 60%, about 70%, about 80%, about 90%, about 100%, about 200%, about 300%, about 400%, about 500%, about 600%, about 700%, about 800%, about 900% or about 1000%, compared to the clotting time of blood isolated from the otherwise similar subject without platelet dysfunction.

33. The method of claim 15, wherein the closure time of platelets isolated from the first subject before administration of meloxicam is prolonged by about 1% to about 1000%, compared to that of the closure time of platelets isolated from the otherwise similar subject without platelet dysfunction.

34. The method of claim 33, wherein the closure time of platelets isolated from the first subject before administration of meloxicam is prolonged by about 1%, about 5%, about 10%, about 20%, about 30%, about 40%, about 50%, about 60%, about 70%, about 80%, about 90%, about 100%, about 200%, about 300%, about 400%, about 500%, about 600%, about 700%, about 800%, about 900% or about 1000%, compared to the closure time of platelets isolated from the otherwise similar subject without platelet dysfunction.

35. The method of any one of claims 1-34, wherein the closure time of platelets isolated from the first subject after administration of meloxicam is comparable to a closure time of platelets isolated from the first subject before administration of meloxicam.

36. The method of any one of claims 1-35, wherein the closure time of platelets isolated from the first subject after administration of meloxicam is comparable to a closure time of platelets isolated from a second subject, wherein the second subject has the platelet dysfunction, and wherein the second subject is not administered meloxicam.

37. The method of any one of claims 1-36, wherein the clotting time of blood isolated from the first subject after administration of meloxicam is comparable to a clotting time of blood isolated from the first subject before administration of meloxicam.

38. The method of any one of claims 1-37, wherein the clotting time of blood isolated from the first subject after administration of meloxicam is comparable to a clotting time of blood isolated from a second subject, wherein the second subject has the platelet dysfunction, and wherein the second subject is not administered meloxicam.

39. The method of any one of claims 1-38, wherein the closure time of platelets isolated from the first subject after administration of meloxicam is at least about 10% to about 100% less than a closure time of platelets isolated from a second subject, wherein the second subject has the platelet dysfunction, and wherein the second subject is administered 15 mg/mL of ketorolac.

40. The method of claim 39, wherein the closure time of platelets isolated from the first subject after administration of meloxicam ranges from about 40% to about 50%, less than a closure time of platelets isolated from a second subject, wherein the second subject has the platelet dysfunction, and wherein the second subject is administered 15 mg/mL of ketorolac.

41. The method of any one of claims 1-40, wherein the clotting time of blood isolated from the first subject after administration of meloxicam is at least about 5% to about 100% less than a clotting time of blood isolated from a second subject, wherein the second subject has the platelet dysfunction, and wherein the second subject is administered 15 mg/mL of ketorolac.

42. The method of claim 41, wherein the clotting time of blood isolated from the first subject after administration of meloxicam is at least about 5%, about 10%, about 20%, about 30%, about 40%, about 50%, about 60%, about 70%, about 80%, about 90%, or about 100%, less than a clotting time of blood isolated from a second subject, wherein the second subject has the platelet dysfunction, and wherein the second subject is administered 15 mg/mL of ketorolac.

43. The method of any one of claims 1-42, wherein the meloxicam is administered in a volume of about 1 mL.

44. The method of claim 40, wherein the closure time of platelets isolated from the first subject after administration of meloxicam is about 44% less than a closure time of platelets isolated from a second subject, wherein the second subject has the platelet dysfunction, and wherein the second subject is administered 15 mg/mL of ketorolac.
